(19) United States
(12) Patent Application Publication
(10) Pub. No.: US 2020/0066414 A1
(43) Pub. Date: Feb. 27, 2020

(54) TELEHEALTH SYSTEMS

(71) Applicant: THOMAS JEFFERSON UNIVERSITY, Philadelphia, PA (US)

(72) Inventors: Robert NEFF, Villanova, PA (US); Judd HOLLANDER, Villanova, PA (US); Neil Dominic GOMES, Moorestown, NJ (US)

(73) Assignee: Thomas Jefferson University, Philadelphia, PA (US)

(21) Appl. No.: 16/344,611

(22) PCT Filed: Oct. 25, 2017
(86) PCT No.: PCT/US2017/058336

§ 371 Date: Apr. 24, 2019

Related U.S. Application Data
(60) Provisional application No. 62/412,616, filed on Oct. 25, 2016.

(51) Int. Cl. G16H 80/00; G16H 10/20; G16H 10/60; G16H 40/63; G16H 40/67; H04L 12/18; H04L 12/58; H04M 3/432; H04M 3/42

(52) U.S. Cl. CPC G16H 80/00; G16H 10/20; G16H 10/60; G16H 40/63; G16H 40/67; H04M 2201/40; H04L 51/046; H04M 3/432; H04M 3/42221; H04M 2203/1016; H04M 2201/50; H04L 12/1818

(57) ABSTRACT

The system comprises a control module, wherein a medical professional connects to a patient to initiate a telehealth appointment. In certain embodiments, the system comprises a control module accessible to a medical professional, software or an app loaded on the control module and on a second user (patient) device. By running the software or app on both devices, the medical professional and the patient can initiate a connection between the two devices.

### CROSS-REFERENCE TO RELATED APPLICATIONS

This application claims priority to U.S. Provisional Patent Application Ser. No. 62/412,616, filed Oct. 25, 2016, which is hereby incorporated by reference in its entirety.

### FIELD OF INVENTION

The present invention is generally related to systems for enacting and optimizing telephonic and video systems for communicating between a doctor or medical professional and a patient.

### BACKGROUND OF INVENTION

Telemedicine is a recent medical care strategy where patients and medical providers can utilize now ubiquitous mobile phones, smart phones, and internet capabilities to hold telephonic and video capable care sessions in order to provide for immediate patient care, and greater access to patients and medical providers. A benefit of the system is that a patient has the ability to meet with a medical professional from the comforts of their own location, whether this is home, the office, or another location, and still receive medical evaluation and prescription of medications to treat afflictions. Furthermore, there are possible efficiencies for a medical practitioner of reducing the size of space necessary to meet with patients, or having the ability to meet with a remote client without forcing either party to travel. Finally, remote meetings allow a sick patient to remain quarantined away from other patients yet still meet with a medical practitioner to receive evaluation and treatment.

As with any new technology, problems arise and solutions are needed to improve the technology. For example, known issues include the quality of the voice or video connection; the ability to manipulate a telephone for video or photographic presentation; switching to and from voice or video calls; assisting or controlling a patient's device to enable visualization of the patient or areas for treatment; advertisements and/or product placement; professional nature of the call; and other issues.

Solving one or more of these problems would improve the quality of the services provided in a telehealth setting and improve the efficacy of treatment able to be provided.

Certain publications have attempted to solve one or more of the issues identified herein, yet each contain strategies than are difficult to implement, or omit essential and novel features that would improve their functionality in a professional setting.

For purposes of connecting to a telemedicine call several patents or publications provide a basic framework for such a call. For example, U.S. Pat. No. 6,027,217, US 2005/0251421, US 2006/0064319, U.S. Ser. No. 12/636,390, US 2011/0090303, US 2011/0218410, US 2011/0106557, US 2011/0213625, US 2013/0035955, US 2014/0249878, US 2013/0246084, US 2015/0127381, 2015/0180917, US 2015/0261918, US 2015/0331997, US 2016/0104486, and EP 2892428. However, these basic overview patents, do not solve or even consider many of the issues that continue to plague telehealth appointments.

### SUMMARY OF INVENTION

In the broadest sense, described herein are systems for enacting a telehealth call between a medical provider and a patient, and methods of providing an improved telehealth call, wherein the system provides for the ability to connect between medical provider communication device and a communication device from the patient; wherein the system allows the medical provider to connect to the communication device from the patient so as to control the electronic systems therein; wherein the system can then provide feedback to the patient to orient the device during the call; further, the system is able to provide real-time feedback to the medical provider about certain quality control features; finally, the system can receive inputs from an EMR, or through voice of video during the call for targeted advertising to the patient and/or medical provider.

A method for performing quality control procedures on a telehealth call comprising: connecting a control module from a first user to a device of a second user to generate a voice or video connection between the control module and the device; performing an analysis of the voice of the first user to determine proper tone for the connection; performing an analysis of the background of the first user; providing feedback to the first user based upon the analysis of the voice and of the background.

Systems of the preferred embodiments may further include one or more of the following features:

One Click Doctor Visit.

As soon as the app is launched, an intake coordinator, or clinician is available via video. This reduces any barriers to the patient calling/connecting. They don't need to remember passwords, accounts, or other information. In some cases an intake specialist will collect name/insurance/allergies, etc. from the patient and then do a warm handoff to clinician. In some cases the one click process will start after the login.

Advertising During Telehealth.

There are already opportunities to advertise in apps, but in this case we will be advertising throughout the telehealth workflow. Some companies have considered advertising in a telehealth virtual waiting room, but that ideas lacks the true value of being able to target advertisements based on patient conditions. This invention includes advertising through scrolling marquees and banners during the patient visit, post the patient visit, throughout follow up emails sent to the patient, and embedded in the discharge instructions delivered to a patient. All such advertisements will have the ability to be targeted for specific patients through data which was obtained during the visit, or during prior visits. Data will be pulled from the EMR, and potentially in real time through voice analysis and natural language processing of the discussion with the provider. Delivery of advertisements for relevant clinical trials can be a major focus of such advertising.

Warm Handoffs.

Warm handoffs have become increasingly common with voice phone calls in call centers. Expanding this concept to telehealth and providing warm handoffs in the case of video calls is even more important. Discussing health is extremely personal, and it is already challenging to shift that discussion to electronic mediums. By providing warm handoffs between practitioners, the patient is afforded extra comfort with the technology that is imperative when delivering healthcare via this medium.

Convert Voice to Video.

The ability to convert a standard phone call from voice only to video is a new advantage in the world of telehealth. Right now, the process to initiate a telehealth call is fairly cumbersome. A simple button press during a regular voice call, which initiates a video call is a great step is removing barriers to this technology. There are numerous ways that technology can facilitate this idea. In the simplest example, while on a voice call and a patient is trying to schedule an appointment, they could be asked to press 2 to initiate a video call with a provider instantly. Upon pressing 2 they would see an incoming video call and be able to start speaking to that provider immediately. That provider will also be able to access all the context which was shared during the audio only portion of the call. In other examples, while discussing a concern with a clinician or provider via phone, the provider could switch the call to video to enhance the discussion and care provided.

Post Call Scheduling—

Perhaps the most important part of any patient and clinician interaction is the follow up care. Telehealth lends itself well to having discrete calls, but does not necessarily prompt that patient to seek that follow up care that they should. Embedding a workflow at the completion of a telehealth call that requires (or pushes very hard) for a patient to schedule a follow up visit it required. This follow up visit could also cause a monetary reward (slight discount in cost of visit) if successfully scheduled. There are a few ways in which the follow up scheduling could happen. At the end of the call, the patient could be shown a list of times for follow up appointments, and the patient could schedule online. The patient could also be transferred automatically to our call center to schedule a follow up via voice. Or a facility might employ telehealth visit coordinators, in which case the patient might be given a warm handoff to someone who schedules their follow-up via video.

Targeted Coupon Codes.

Getting patients to seek the care they need is extremely challenging. Offering incentives, via coupon codes (or other discounts) is one tool which can be used to incent patients to get such care. In many cases, offering discounted, or free follow up or preventive care is less expensive than offering nothing and waiting for a medical problem. In this invention, discounts are targeted to patient based on their geolocation, medical conditions, and time since last visit. A patient with a chronic condition who has not been seen in two years may be given more of a discount (even to the point of given a positive reward) for completing a telehealth visit whereas a similar patient who has been seen within the year might only be given/offered a smaller discount/incentive to participate in a call.

Dynamic Surveys.

Automatically sending a survey to determine the quality, level of care and satisfaction that a telehealth visit has is crucial to optimizing the care delivered through this medium. Different patients (based on geographical location or severity and type of medical condition) might need to be asked different follow up survey questions. A patient local to our facility might be asked about follow-up care in our facility, whereas a patient not geographically proximal to our location may not.

Transferring Calls/Patients.

A significant challenge in telehealth today is that licensing restrictions require providers to be licensed in the same physical location of the patient. This restriction poses a challenge for telehealth systems, as the patients can easily call from any location. The invention here allows that patients can be handed off (via hard or cold hand off) to partner telehealth centers which may be on different or separate telehealth platforms or solutions, based on the needs of the patient. A patient in Florida might reach a clinician via telehealth which is only licensed in New York. This clinician would then have an easy workflows to hand that patient off to a clinician which is licensed in Florida. Additional, handoffs could take place to facilitate matching specialists, preventing conflicts of interest, or other reasons. The handoff process would allow sharing of medical records and referral fees where appropriate.

Remote Control.

Today, during a telehealth call, the clinician is to a large extent at the mercy of the settings and operation of the device (likely phone) that a patient is using. A patient might not be aware or technically capable of leveraging all of the phones capabilities to provide the best experience possible. In this invention, the clinician on the call has the ability to manipulate the settings and function of the patient's phone during the call. This includes remotely turning on the flashlight on the phone, adjusting the microphone volume, switching to the back-facing-camera, etc. This can all be done (with the patient's permission [opt-in]) by the provider from his or her console. The console would show options to optimize the phone settings through a simple interface—i.e. the clinician could press a button and it would turn on the flashlight on the phone, regardless of the phone model.

Autoalign.

Adjusting a device so that it providers a clinician the best angle of view is important to the clinician and may be easy for a clinician—but challenging for a patient to understand. Using the gyroscope and other sensors on a device/phone, the clinician can instruct that the patient move the phone into a particular position in 3D space. The process would have the clinician choosing a particular procedure and then the phone guiding the patient to position the phone in accordance with the requirements. The phone might beep and show arrows of positioning information on the screen all in an effort to guide the patient to align the phone properly. This method also ensures that clinicians always views similar medical conditions in the same manner across patients. Aligning the phone so it shows a straight head on video of a sore throat at a known angle is one example of a use case for this such invention.

Recording Discharge Instruction.

Sending a discharge instruction packet is an important part of any clinical visit. However, having the specific words spoken by the clinician available for replay is an additional benefit. In this invention, the patient and clinician can jointly opt in (at the end of the visit) to have the visit summary discussion recorded (either video or just audio) and available for replay at a later time. This recoding may also only be available for X period of time.

Video Overlay.

Providing relevant and timely information about a patient to the clinician is crucial. In the case of telehealth, there is an opportunity to overlay such information on the video stream to give it extra attention for the clinician. Some such information may include (based on geolocation and medical condition) public health information—such as recent outbreaks of disease. Video overlay of common questions to ask the patient for given conditions may also be used.

RealTime Clinician Feedback.

Ensuring the patient has a positive experience is as important as ensuring the clinician can successfully deliver care. In this invention, the clinician is offered automatic feedback about his or her appearance and performance during the visit. The feedback can range from reminding the clinician to focus on the patient (based on tracking clinician's pupil/eye position and focus) to noticing the tone of the voice of the clinician, to commenting on the attire and general look of the clinician.

Visual to Audio Translation.

As telehealth technology progresses and we move to even more advanced remote visualization technology, there will be a time where you can remotely view a holographic representation of a patient. In such a case, using tools such as a stethoscope might seem helpful, but are not technologically compatible—or needed. However there will be a learning curve as clinicians being able to treat visually via a hologram (and inside the hologram) vs. listening to what is happening inside is quite a different art. This invention uses video analysis to recreate the sounds (of heart beating, or lungs exhaling) based on video of patient. This puts the information needed for diagnosis back into a format that the clinician is used to.

The embodiments described herein may use any one or a combination of features to facilitate a telehealth system that provides for improved doctor to patient opportunities.

A preferred embodiment is directed to a system comprising a central hub, a control panel electronically connected thereto, a first communication device and second communication device; wherein the control panel is enabled to control at least one feature of the first communication device, wherein the central hub generates a voice or video connection between the first and second communication devices, and wherein the control panel automatically optimizes the second communication device.

In a preferred embodiment, a telecommunications system for connecting a central hub, a control panel and at least two communication devices; said central hub comprising a database of key words, phrases, and engaged to a software platform for electronically connecting the central hub to the control panel and the at least two communication devices; said first and second communication device having said software platform running on said device; wherein said second communication device generates a video communication request to said server, wherein said software identifies the second communication device and an electronic medical record connected to said software running on said second communication device; wherein said first communication device is notified of said video communication and said central hub generates a control panel, accessible on said first communication device, identifying the features of the second communication device; wherein the first and second communication device generate a video communication, and said control panel is utilized to optimize the sound and video quality by increasing or decreasing the sensitivity of microphone, increase or decrease the volume played by a speaker in each device, and increasing or decreasing the light contrast of the video camera to a pre-defined parameters.

In a preferred embodiment, said systems further comprising a real time monitoring of the first communication device, said real time monitoring comprising optimizing the parameters of the first communication device, providing feedback to the provider to modify one or more actions, and optimizing parameters of the first communication device.

In a preferred embodiment, said systems further comprising wherein the real time monitoring provides feedback on the control panel to automatically or manually modify one or more parameters on the first or second communication device.

In a preferred embodiment, said systems further comprising a video overlay which is displayed on said control panel, said video overlay providing geolocation of the second communication device.

In a preferred embodiment, said systems further comprising wherein said central hub is capable of recording voice or video, and said control panel capable for engaging recording status; and recording voice and video of instructions to a patient. In a preferred embodiment, the system further comprising wherein the recording of voice and video of the instructions to a patient are transcribed from a recording to text.

In a preferred embodiment, said systems further comprising wherein the control panel is engaged to control one or more features on the second communication device; wherein the control of one or more features engages with a gyroscope on said second communication device to orient said communication device into a desired position. A preferred embodiment comprises further engaging the accelerometer or at least one camera on said second communication device. In a preferred embodiment, the system further comprising wherein the second communication device generates sounds or visual cues for orienting the second communication device into said desired position.

In a preferred embodiment, said systems further comprising wherein a communication is generated to a third communication device, based upon need or state licening requirements to provide medical services.

In a preferred embodiment, said systems further comprising wherein the central hub is capable of detecting speech and for identifying key words or phrases related to medical care; wherein identification of key words or phrases are combined with an electronic medical record (EMR) having codes corresponding to medical information is accessed by said server, and said key words or phrases and said EMR are utilized to generate a survey.

In a preferred embodiment, said systems further comprising wherein the central hub is capable of detecting speech and for identifying key words or phrases related to medical care; wherein identification of key words or phrases are combined with an electronic medical record (EMR) having codes corresponding to medical information is accessed by said server, and wherein a coupon code is generated in response to the key words or phrases and said EMR. In a preferred embodiment, the system further comprising a database stored on said central hub of coupon codes, wherein the key words or phrases and the EMR are compared to the database of said coupon codes for identifying coupon codes for deliver to the second communication device.

In a preferred embodiment, said systems further comprising wherein the server generates an automatic message to the second communication device for scheduling a secondary call.

In a preferred embodiment, said systems further comprising wherein a voice connection is first generated and wherein the control panel is engaged to modify the connection from a voice connection to a video connection; wherein the control panel and server generate a signal to the first communication device and the second communication device to activate a camera and engage video mode.

In a preferred embodiment, said systems further comprising wherein the central hub is capable of detecting speech and for identifying key words or phrases related to medical care; wherein identification of key words or phrases are combined with an electronic medical record (EMR) having codes corresponding to medical information, accessed by said server, and said server further comprising a database comprising advertisements corresponding to one or more key words, phrases, or codes in an EMR, and wherein said key words or phrases, or EMR codes are compared to said database comprising advertisements, and wherein said advertisements are delivered to said second communication device.

A preferred embodiment is directed towards a method of optimizing sound, and video parameters on a telehealth video call comprising: generating a video connection between a central hub, a control panel, and a first and second communication device; reviewing the parameters for sound, by comparing the received sounds from the first and second communication devices to a feedback level standard; comparing the video parameter to a predetermined video feed quality; modifying one or more of a microphone sensitivity, a video sensitivity, a camera zoom, a camera black and white balance, and speaker volume to modify the parameters; reviewing the modified parameters and compare to the feedback level standard and predetermined video feed quality; and proceeding with said video call, while repeating steps b and c automatically during said call to maintain said optimized settings.

In a further embodiment, said method wherein a parameter can be manually modified by the first or second communication device.

In a further embodiment, said method wherein the control panel can modify the second communication device manually.

A further embodiment is directed towards a method of orienting a mobile device to a predetermined position for capturing an image comprising: connecting said mobile device to a central hub system; said central hub system comprising a central hub, a control panel, and a first communication device; providing access to said hardware and software controls on said mobile device, through a software application installed therein; wherein said software application engages said control panel to identify features of the mobile device capable of control; identifying a predetermined position on said control panel; generating a geolocation of said mobile device using one or more of the gyroscope, accelerometer, or camera; providing an oral or visual instruction to modify the present position of said mobile device to said predetermined position; providing an oral or visual instruction once said predetermined position is located; capturing an image from said camera at said location; and uploading said image to said central hub.

A further embodiment, said method wherein said image is provide for determining a medical diagnosis.

A further embodiment, said method further engaging a flash or light on said mobile device when capturing said image from said camera.

### BRIEF DESCRIPTION OF THE DRAWINGS

FIG. 1 depicts a flow chart of the telehealth system.

FIG. 2 depicts an embodiment of a telehealth system comprising a server, control panel, a first and second communication device, interconnected to control and optimize the components.

FIG. 3 depicts an embodiment of components of the telehealth system described herein, particular the control panel, server, and consumer device, and their interaction.

FIG. 4 depicts a flowchart of enacting remote control of a consumer device through the telehealth system.

FIG. 5 depicts a flowchart of enacting auto align of a consumer device to capture sound or images of the patient.

FIG. 6 depicts a flowchart of providing feedback to a provider during a telehealth call.

FIG. 7 depicts a flowchart of generating a hologram or audio representation of a patient.

FIG. 8 depicts a flowchart of providing emergency assistance to a patient during a telehealth session.

FIG. 9 depicts a flowchart of a one-click visit in a telehealth session.

FIG. 10 depicts a flowchart of a warm handoff in a telehealth session.

FIG. 11 depicts a flowchart of generating a voice to video call.

FIG. 12 depicts a flowchart of transferring a call from one provider to another.

FIG. 13 depicts a flowchart of providing discharge information to a patient.

FIG. 14 depicts a flowchart of generating a survey for a patient.

FIG. 15 depicts a flowchart of generating coupons for a patient.

FIG. 16 depicts a flowchart for scheduling a future telehealth session.

### DETAILED DESCRIPTION OF THE EMBODIMENTS

Today, during a telehealth call, the clinician is to a large extent at the mercy of the settings and operation of the device (likely phone) that a patient is using. A patient might not be aware or technically capable of leveraging all of the phones capabilities to provide the best experience possible. In this invention, the system enables a clinician on the call to manipulate the settings and function of the patient's phone during the call and to optionally perform one or more of the additional features as described herein.

The system and invention described herein provides for an improved system to generate and facilitate voice and video communication between a medical professional and a patient. The term “medical professional” includes doctor, nurse, medical assistant, or other caregiver who is in the business of providing medical services or support to a patient. The patient may be any person who is seeking or receiving these services.

A preferred system therefore comprises a first control module controlled by a first user, typically a medical practitioner. A second user and a second user device, typically a smart phone or an internet connected device. The control module and the second user device connect to other another to generate the telehealth appointment.

A new patient may follow the following general path to initiating the telehealth call. The user downloads an app, a medical professional has a control module having the app installed. The medical professional or the patient initiates a telehealth appointment and the control module is connected to the patient's device. The medical professional is able to control the user's device to assist with the appointment evaluation.

The system can take a voice call and turn the voice call to a video call, with the push of a button, or—alternatively, end the video and go to voice only. For example if someone calls and identifies certain issues, the physician can request change to video to view the symptoms.

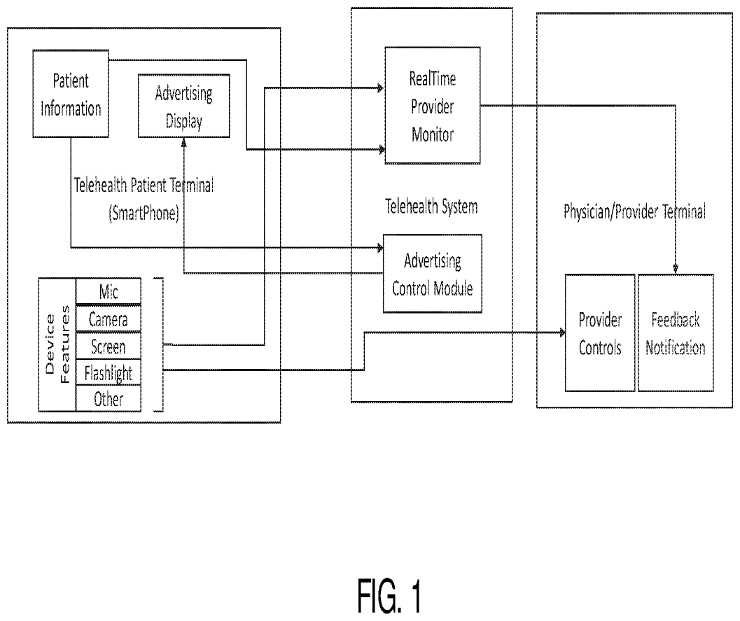

Therefore, as depicted in FIG. 1 the system comprises a control module (Physical/Provider Terminal) having a provider control and feedback notifications. The system connects the control module to a “Telehealth Patient Terminal” typically a patient's smartphone or other mobile or web based access device. The provider controls are based upon the actions capable on the User's device, as depicted on the bottom of the left side of the User's terminal image.

The System (depicted centrally) can monitor in real time the controls, as well as the physical and provide a feedback notification (right side) to the physician. Furthermore, the system can utilize the information from the patient as well as the realtime aspects to identify and display certain advertising to the patient. This allows the system to centrally communicate between a patient user and the medical professional user.

In certain instances the medical professional and the User each have a smartphone device and each run an application to connect to the Telehealth system. Through accessing the Telehealth system, each provides access or allows control of their device through the system to facilitate the telehealth call as described in the embodiments herein. The options to proceed can be individually optimized in the system, or the system can utilized one or more of the options to facilitate and improve the telehealth call.

These features are described in greater detail in the following embodiments. It is specifically noted that the system and methods described herein are modular. That is components of one system or one method are advantageously combined with the others, as necessary to generate the necessary technical effect. Those of skill in the art will recognize that the features and systems described herein will utilize certain common components to enable certain features, and thus specifics of these features are omitted where appropriate. In many instances, the connection procedures are similar to a certain point, wherein a further action is necessary and thus an embodiment can be utilized at that point. Therefore, the embodiments are fully interchangeable with one another so as to generate a system and methods using such systems for enacting telehealth medical appointments.

Throughout are referred to telehealth “sessions” or “calls” which are a voice only call or a video call, comprising voice and video. On occasion, a voice only call may exist, but such is still a telehealth session or call.

Connecting to the Telehealth System

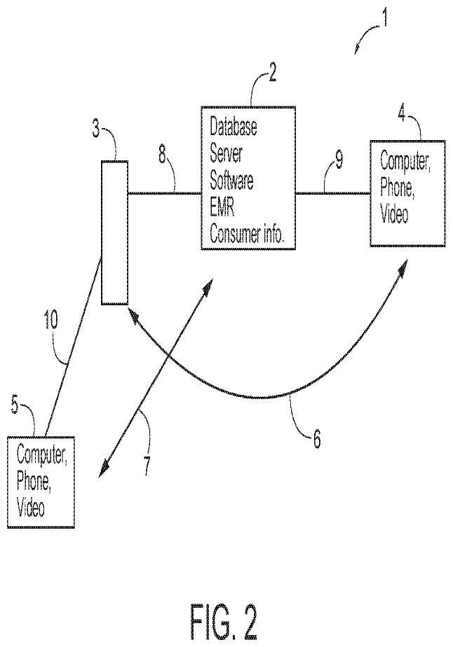

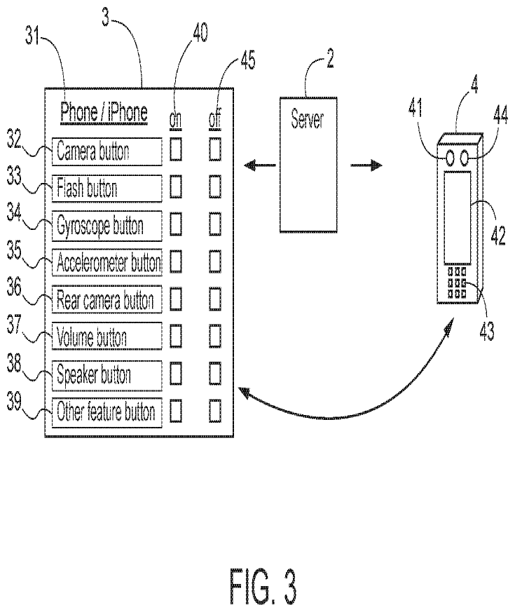

FIG. 2 depicts a communication system 1 for communications via voice or video between a provider device 5 and a user device 4. The system comprises a central processing hub 2, such as a server, and comprising a database entry system, software, processor, memory, and the like, necessary for running such software. This central processing hub 2 has access, through digital channels to an EMR of a patient, as well as the consumer's information. The central processing hub 2 is electronically connected (direct or wirelessly) to a control panel (module) 3, having display capabilities, as well as further features, as described below in FIG. 3. The control module 3 is connected to the practitioner device 10 for control and modification of the practitioner device 5. Similarly, the practitioner device 5 is connected to the central hub 2 via an electronic connection 7, to facilitate call, video or modifications through the provider side features, i.e. the provider device 5 and control panel 3. The control panel 3 connects to a patient device 4, with an electronic connection 6, to facilitate understanding of the features of a consumer device 4, as well as modify and control said features as provided in the embodiments herein.

The telehealth system 1 utilizes these primary components to modify and control one another, automatically or manually, to enhance the telehealth call, video call, or session including both voice and video. In certain embodiments, the system 1 comprises a control module 3 accessible to a medical professional 6, software or an app loaded on the control module 3 and on a second user (patient) device 4, allow communication and facilitation through the central processing hub 2. By running the software or app on both a provider device 5 and a patient device 6, the medical professional and the patient can initiate a connection between the two devices and allow and facilitate electronic modification and improvement of the telehealth appointment.

In certain cases, the app may generate a voice call between the medical professional and the patient. While voice only calls may be useful in certain situations, the ability to convert a traditional voice call to a video call by pressing a button during the call to initiate the video call increases the value of the telehealth appointment. For example, during a voice call, the patient wishes to elect a video call and can press a button, which will initiate a video call instantly. Upon pressing said button, the system initiates a video call, wherein the caller can transfer or hang up the voice call and accept the video call.

In other embodiments, during a voice call, the provider or practitioner can switch the call from voice only to video by election of a button during the call to improve the care of the patient. Furthermore, another embodiment may use a language detection system to identify key words that then can suggest a video call to the participants, or initiate the video call, which the participants can elect to use.

Furthermore, after a video call, the reverse can occur, wherein the video call is terminated and a voice call is activated so as to schedule additional follow-up care.

Therefore, the system 1 may comprise a control module 3 comprising a central hub 2, software executable on said computer and at least one other voice and/or video enabled device, wherein the control module 3 facilitates a call or video between the medical professional on one hand and a patient on the other hand, and can interchangeably direct a voice to video or video to voice, based on the needs of the users on the call. Further embodiments may be directed to methods of telehealth patient care including the features of switching between voice and video based on the needs of the participants. Each embodiment may also utilized automatic transfer from voice to video or video to voice based upon certain key works, or based up on election of one or both of the parties on the call.

In certain embodiments, there may be a delay between the initiation of a voice or video call. A patient may first dial into a telehealth appointment, yet the medical professional is not yet available to connect on the other end. This creates a virtual waiting room, wherein the patient may be provided with any number of features including information about the purpose of the call, general health information, advertisements, and the like.

Control Between the Control Module and Patient Device

In a telehealth appointment, the central components of telehealth system 1 comprises control module 3 and a central hub (server) 2. The control module 3 may be a computing device, having a processor, memory, storage, a screen, and capable of running a software program. Similarly, the central hub or server 2, facilitates communication between and among the components, including the above features as well as a patient device 4 and a provider device 6, or provider devices, as may be necessary to provide care in the telehealth system. The Server, as known to those of skill in the art must have sufficient components to run the software and provide for instructions and communications between and among the several components, and thus comprises one or more processors, memory, storage, data basing systems, and capable of running software. Together, these components facilitate the pairing between a consumer device 4 and a provider device 5 of a voice or video call. A device is typically a mobile phone, though electronic communication can be further provided by wired phones, internet based calls or videos, or other systems as necessary and able to electronically connect. Devices for speech or hearing impaired can include both sound and words, voice or video, or all, to facilitate communication between the parties.

Upon initiation of a call or video call from a patient device 4, connection is generated to the central hub 2, which, when the call is using software or application recognized by the central hub 2, will connect the patient device 4 to a third party. However, simultaneously, between the control module and the patient's device, the control module 3 accesses the patient's device 4. For example, FIG. 3 provides a detail of this connection, with the central hub (server) 2 between the patient device 4 and the control module 3. The patient device 4 comprises a patient device camera 41, a patient device flash 44, a patient device display 42, and a patient device keypad 43, among other features of an ordinary smart phone device. Indeed, not shown externally are additional components, including a gyroscope, an accelerometer, as well as a rear camera and/or rear flash. Other features of the device may also be present. The control module 3 identifies the type of the device 31, and then provides on the display of the control module 3 a set of features of the device. In this example, the device is listed as an iPhone, and access is provided to the camera 32, flash 33, gyroscope 34, accelerometer 35, rear camera 36, volume 37, speaker 38, and an “other feature” 39. Intuitively, there is an “on” 40 or “off” 45 feature for many of these devices. For example, the camera can be turned on or off, and then appropriately zoomed or focused, for example with the “other feature” button controlling those particular features. The Flash 33 can be on or off, as necessary to illuminate the patient or a feature. The gyroscope 34 too can be enabled to orient the patient's device 4, and communicate its relative position in space. The accelerometer 35 can detect movement and motion, often used in conjunction with the gyroscope 34. The rear camera 36 enables a rear or second camera on a device. This allows for visualizing from another camera, and/or helps to orient the device, the patient, or provider. For example, both cameras activated together may provide a better 3D visualization, or help, with the gyroscope 34 to orient the device in space to allow for orienting the camera to take an image. The volume button 37 can increase or decrease the volume received by the microphone, thus increase or decreasing the sensitivity of the speaker. Thus, if the sound received by the patient device 4 is too loud, the volume can be decreased, if the speaker is speaking too quietly, the sensitivity can be increased to pick up more sound. This allows the provider to optimize the call parameters. Similarly, if the patient is having trouble hearing, the speaker button 38 can increase the sound from the speaker in the handset of the patient device 4. Thus the speaker volume can be increased or decreased as necessary to optimize. Finally the other feature button 39 is activated by any number of devices or options that become available by activating one or more of the features 32-38, or by a feature not otherwise listed herein. There may be several “other feature” buttons 39 available, if multiple features or components have further capabilities. Indeed, the control panel 3 connects with the software and hardware of the patient device 4 to allow for modification of and control of the features of that device. This enables the medical professional to access to options to control the patient's device 4 and optimize said features.

For example, the patient has a smart phone 4 that can connect either with a voice only call or with a voice and video call. Furthermore, the patient's phone 4 may be able to take photos or video, and to zoom in and out, as well as use a light or flash to illuminate a space. The control module 3 thereafter provides the medical professional with these options in order to improve the quality of the telehealth appointment.

In certain embodiments, the system requires accessing or providing control features from the clinician to the user device, and then providing instructions with visual, audio, or tactile (vibrations) stimuli. Accordingly, the system allows a medical provider to connect to a user device (phone), wherein the user device receives input based on the gyroscope and other sensors within the user device to instruct the user to move the user device in a particular orientation in 3D. For example, the phone may flash or beep, or give verbal instructions on how to orient the phone according to the desired positioning from the physician.

There are several reasons why this is beneficial to telemedicine. For example, certain users may not be able to visualize or orient their phone according to the directions from the clinician directly. However, the program, using lights or beeps, or other visuals on the screen, may assist the user with placing the phone in the proper orientation. Another reason is that clinicians may be able to orient the phone to a consistent position for imaging, and thus consistent images across an individual patient (i.e. follow-up calls), or across many patients, may assist with providing consistency of care in the telemedicine setting. The Proposed Invention requires accessing or providing control features from the clinician to the user device, and then providing instructions with visual, audio, or tactile (vibrations) stimuli. This can include lights, flashes, or images on the screen of a device for the visual, sounds like beeps or spoken instructions, and vibrations. Wherein the position and instructions are modified based on the orientation from sensors within the phone to provide for the proper positions for the clinician. In order to provide these features, there must be an ability to control the features of the patient's device.

Remote Control

A further application of the system 1 and the ability to control a patient's devices 4 is that some users do not know all of the functionalities of their telephones, or how to appropriately utilize or access all the features of their device. For example, not all users know how to switch between the different cameras on the device or accessing the flashlight feature. However, for the purposes of a telehealth video call, a medical practitioner may need the patient to be able to manipulate the mobile device 4 to be oriented in a certain manner and to utilize a light or camera to view the issue that is being addressed in the telehealth call. For example, a patient may have soreness in the throat, and the medical practitioner would like the patient to use the rear facing camera and the flashlight feature together to shine within the throat. However, where the patient cannot make these changes herself, access and control of these features is required to assist the medical practitioner in examining the patient remotely.

Accordingly, the system 1 provides for connection between the control module 3 and a user's device 4 so that the device 4 can be remotely controlled by the medical practitioner. Thus, during the telehealth session, the medical practitioner has the ability to modify the phone 4 controls remotely. The preferred embodiment would utilize an application 14 on the phone 4, wherein access is granted to the medical practitioner. Then, the application would connect the mobile device 4 to a central computer controlled system 2, wherein the medical practitioner can modify the mobile device through a control panel 3 in the central computer controlled system. Therefore, the medical practitioner is able to turn on the flashlight, switch the camera being utilized, increase volume of the phone speaker, etc. The system would generate a connection to the phone and a display of the possible features to modify would be presented to the medical practitioner. Thus, phones of different vintage and capabilities would only identify the features that could be modified.

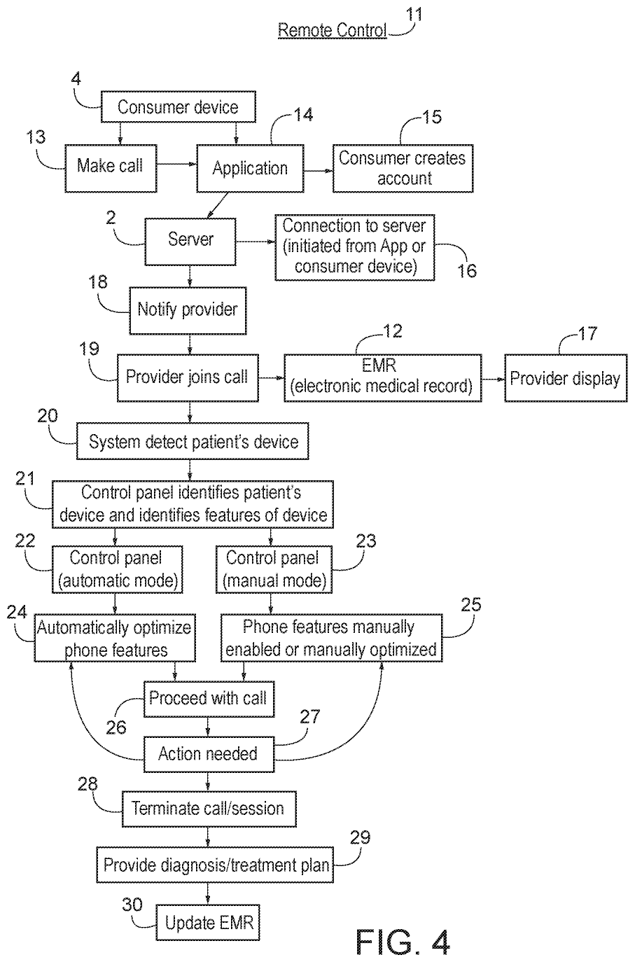

FIG. 4 provides a flow chart of a connection to the system 1 and enacting remote control 11 over the patient's device 4. The consumer device 4 is depicted at the top, wherein a consumer initiates a call 13. If the consumer has the telehealth application 14 installed, the call is placed through the application. The patient/consumer creates an account 15. The account requires patient information, username, password, and the like. By creating an account, however, the application can then access the EMR of the patient, and this facilitates access to and updating of the EMR for future reference.

Through the application 14, the call connects to the server/central hub 2, which then, notifies a provider 18 and the provider can join the call 19. Simultaneously, the Server 2 generates a profile of the patient on a provider display 17. This provider display can be on the provider's own device 5, a separate computer system, or as part of the control panel 3. Typically, the EMR is accessible, as well as additional information related to the patient that is generated through the application.

The system also detects the patient's device 20, and the control panel identifies the type of the device and features of the device, for example as shown in detail in FIG. 3. The control panel then optimizes the parameters of the call, either automatically 22 or manually, and then the provider and patient proceed with the call. Additional features and control of the patient's device 4 may be necessary, and thus, if an action is needed 27, the control panel can automatically or manually perform such actions, as will be described in further embodiments. At the conclusion, the call or session can terminate 28 with the diagnosis or treatment plan being provided either before termination, or after termination, where appropriate based on the particulars of the session. Finally the provider can update the EMR.

It is important to note that the aspect of this remote control 11 can be combined with one or more of the features and embodiments, and in some cases, it is a necessary step before such embodiments can be completed.

The following is an example of how this remote control 11 feature can be utilized to improve the features of the telehealth session. A patient initiates a call, based on a pre-determined appointment time with her provider. As she makes the call 13, the server 2 connects 16 to the device and notifies the provider 18 that the appointment is ready to proceed. During this time, there may be a slight delay, between the notification 18 and the provider joining 19, and, while not shown here, the patient is essentially “on-hold.” This is a virtual waiting room, in that sense, and the patient may receive information, ads, or other info during this time. Once the provider joins the call/session 19, the system has detected the patient's device 20 and a control panel 3, has identified features 21 of the patient's device. As the patient and practitioner communicate, the control panel is automatically optimizing the features of the phone 24 to ensure that the sound and light are proper. Thus, the automatic steps may use a camera, use a flash, zoom a camera, increase or decrease the sensitivity of a microphone, increase the sound played by the speaker to the patient. For example, the system can pick up key words, such as “I can't hear” and increase the sound from the speaker automatically. At the same time, the provider can manually perform these actions 25. If the patient says “I can't hear” the provider can manually increase the volume until it is proper. The automatic optimization 24 can be switched off, if necessary for manual control.

A particular embodiment of the disclosure is directed to a method of performing a telehealth call comprising the above features.

A particular embodiment is directed towards a method of controlling a mobile device (such as a phone, tablet, smart phone, and including computers), by running an application on said mobile device to allow for communication between said mobile device and a central server or hub. Connecting said mobile device to a control panel, capable of modifying one or more parameters of hardware or software on said mobile device; and connecting said mobile device to a first communication device (i.e. a phone, table, smart phone, computer) of a provider, to provide a telehealth session. The first provider, having access to said control panel can automatically or manually modify the parameters of the mobile device for optimizing the parameters of the mobile device. For example, the features identified in FIG. 3, the camera, flash, gyroscope, accelerometer, rear camera, volume, speaker, or other such hardware features, or control such hardware through software processing. Wherein the method allows for such remote control of said device, particularly for engaging in a telehealth video or voice session.

Today, during a telehealth call, the clinician is to a large extent at the mercy of the settings and operation of the device (likely phone) that a patient is using. A patient might not be aware or technically capable of leveraging all of the phones capabilities to provide the best experience possible. In this invention, the clinician on the call has the ability to manipulate the settings and function of the patient's phone during the call. This includes remotely turning on the flashlight on the phone, adjusting the microphone volume, switching to the back-facing-camera, etc. This can all be done (with the patient's permission [opt-in]) by the provider from his or her console. The console would show options to optimize the phone settings through a simple interface—i.e. the clinician could press a button and it would turn on the flashlight on the phone, regardless of the phone model. Example of operation in this case is if the physician needs to look down the throat of a patient. The physician may request that the patient turns on the flashlight on the phone/camera and also switches to using the back facing vs. front facing camera. The physician will simply push a button on his/her console which then activates the appropriate capability of the phone. Then the patient can simple hold the phone in the position that the physician has requested. The additional value here is not only that the patient need not understand all the capabilities of their device and how to activate them, but the physicians don't need to know how to talk the patients through doing it across the many different devices that exists. The console for the physician will abstract all the capabilities of each device into a simple control panel so that she/he can activate them regardless of the device. Specific devices might not support front and back facing cameras, in which case the physician console will show certain capabilities as not available in some cases.

FIG. 4, while described above in detail is further provided herein, with an overview of certain features and certain optional features. It is noted that certain features may be excluded, wherein they are not needed, or, combined with additional aspects as provided in the further embodiments.

FIG. 4 details “Remote Control” 11, wherein a patient initiates telehealth visit 13 using their device 4;
a. Patient determines he has a medical need now or in the future
b. Patient downloads an app 14 or other digital solution for remote medical video visits.
c. The patient creates an account 15 to record medical information for themselves and their family and dependents and enable login to system/app
d. The patient determines that they have a medical need now and they login to the system/app to initiate a call 13
e. The system/app may include a call center of providers available for calls immediately or at a scheduled time in the future. (the providers may be logged into the system already at this point)
f. The app connects to a server 16 which notifies a provider 18 that a new call/request/visit needs to be serviced
g. Provider joins the telehealth visit 19 and optionally accesses the EMR or records 12 on a display 17
h. The provider accepts the call 19
i. The two way communication session between the patient and provider is initiated
j. Provider or system (automatic mode) 22 optimizes the experience (audio/video)—The system, on automatic mode, can optimize the audio/video experience or the provider have (or take over) manual control 23 modify the remote control options on terminal for adjusting the following remotely on the patient's device:
k. Adjust flashlight on patient phone to compensate for lighting in patient environment. One example might be if the patient has a window behind them making them appear too dark. Adjusting lighting from the front of the patient would facilitate better visualization and examination of the patient
l. Adjust mic on patient phone to optimize clarity of patients voice and screen out background noise
m. Adjust speaker volume on patient phone to optimize patient ability to hear provider(s)
n. Switch camera from front to back to allow patient to easily use back facing camera
o. Allow the provider to access patient phone for full control including adjusting any other settings, including downloading medically related apps that can be used by telehealth provider. An example would be like when an IT help desk accesses the computer remotely. The system has the ability to automatically adjust all of these things for the patient if needed.
p. Initiates screen stabilization for patients who cannot hold the camera steady. An example of this would be patients with certain conditions such tremors;
q. Give provider remote access 20 and 21 to any other sensors that are included in (or connected externally with) the patient's device which could be available for control by the remote provider/physician
i. For example: Hear rate sensors, respiratory rate, O2 sat, any other medical device which is attached to the device/phone, accelerometer, gyroscope (car crashes or fall s/fainting)
r. The patient and provider determine the visit is complete. 28
s. The patient or provider ends the visit/call. This might be done by clicking on “done” or by saying “end call” or the equivalent.
t. The provider completes his documentation and closes the encounter. 30

The features of Remote Control 11 detail how the system 1 enables connection to the user device 4 and some examples of the use of those features. However, the control feature may powerfully utilize the components of the patient device 4 to provide further support to the provider for examining the patient and providing quality care.

Autoalign

Adjusting a device 4 so that it providers a clinician the best angle of view is important to the clinician and may be easy for a clinician—but challenging for a patient to understand. Using the gyroscope and other sensors on a device/phone, the clinician can instruct that the patient move the phone into a particular position in 3D space. The process would have the clinician choosing a particular procedure and then the phone guiding the patient to position the phone in accordance with the requirements. The phone might beep and show arrows of positioning information on the screen all in an effort to guide the patient to align the phone properly. This method also ensures that clinicians always views similar medical conditions in the same manner across patients. Aligning the phone so it shows a straight head on video of a sore throat at a known angle is one example of a use case for this such invention. This system is similar to that which is used in google sky view or other apps where they guide you to position your phone a certain way. Imagine the case of a physician needing to look into your mouth at a specific angle, but you not knowing how to hold the phone. The system can be preprogrammed how to hold your phone/camera for specific conditions (maneuvers). Additionally, the physician may be able to drag or touch the screen to adjust the image and this would adjust the guide arrows on the screen of your device. Finally, in an additional claim the system might use image recognition to determine if the device in positioned appropriately for a particular request (i.e. is the mouth or throat centered on the screen). The physician would initiate the process of AutoAlign through his/her console and then the patient would follow the onscreen instructions (and possibly auditory beeps) to move the phone into proper position. The system may also indicate (through beeping, visual cues, or hepatic feedback) if the patient is straying from the initial position (i.e. with a shaky hand). In another claim, the system may use technology to automatically steady an image so the physician gets a better view—even if the patient is shaking. This feature would be initiated by the physician, because normalization of the image in any way automatically could mask possible clinical conditions affecting the patient.

During a video call, control module allows for the medical professional to utilize a control panel in which she can control the user's phone. The goal of this is to engage the rear camera or the front camera, turn on the flashlight, change the zoom, etc. Autoalign can be utilized from the control module, wherein, after taking control of the user's device, the gyroscope of the device can help to orient the phone to a patient.

This may be advantageous, where a medical professional wants to visualize the patient's throat area. A user, holding her own phone, may have difficulty orienting the phone to properly image the back of the throat, to check for strep throat, for example. Some patients may find it difficult to place the phone in the correct orientation to get the necessary image. The phone can then identify where it needs to go and provide instructions to orient properly. This will allow more consistent images for a single patient, or for multiple patients, when looking at similar issues. This helps consistency of care for one or many patients.

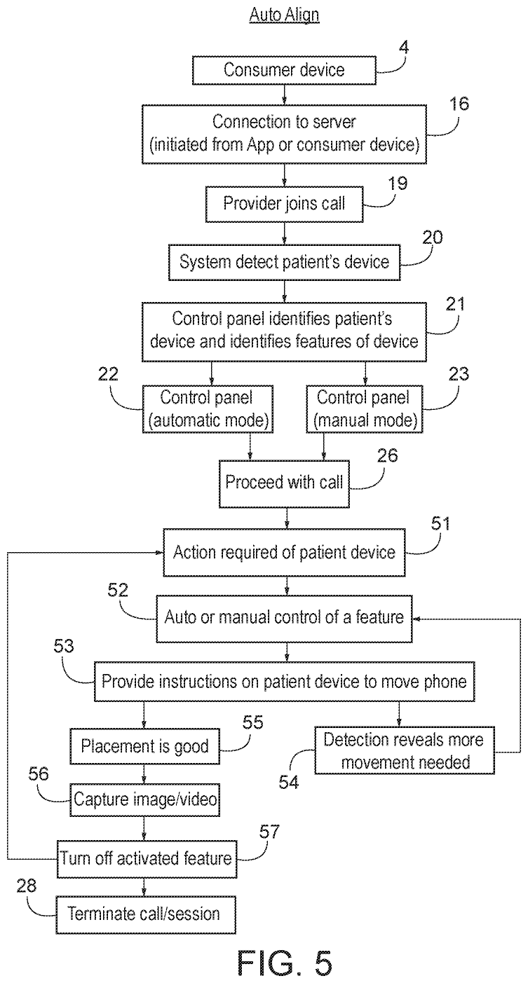

FIG. 5 details the auto align concept with greater specificity. Certain of the features are omitted, or assumed, based upon prior FIG. 4, for clarity as to the specific actions of this aspect the following list is provided.
1.) Patient initiates telehealth visit
   a) Patient determines he has a medical need now or in the future
   b) Patient downloads an app 14 or other electronic solution for remote medical video visits.
   c) The patient creates an account 15 to record medical information for themself and their family and dependents and enable login to system/app
   d) The patient determines that they have a medical need now and they login to the system/app and initiate a call 13—The system/app includes a call center of providers available for calls immediately or at a scheduled time in the future. (the providers may be logged into the system already at this point)
   e) The app 14 connects to a server 16 which notifies a provider 18 that a new call/request/visit needs to be serviced
2.) Provider joins the telehealth visit 19
   a) The provider accepts the call
   b) The two way communication session between the patient and provider is initiated
3.) Provider determines a need to evaluate patient from a specific angle or perspective 51
4.) Provider initiates auto align process to guide the patient to re-orient camera/phone/device which patient is using to the correct perspective/angle, which can be manual or automatic, or combination of the two 52. Provider selects (via mouse click, touch screen, oral instructions, or otherwise) on his screen 17 the part of the patient (area of interest) which he wants a better look at.
a) The system knows that anatomic alignment that is necessary for the specific area of interest of the patient. An example would be looking at the throat of a patient. The system would know that the provider needs to have a view that includes the tongue, palate, tonsils and uvula and would autoalign to get optimal view of this anatomy.
b) The system determines through measurement of pixels, the correct direction which the patient needs to move the phone/camera/device such that the area of interest is centered in the field of view of the camera.
c) The system may use the gyroscope or other sensors to determine correct positioning
d) The positioning mechanism will likely include camera, gyroscope, and accelerometer data in concert to determine the correct position instructions
e) The patient's phone/camera/device will use arrows overlaid on its screen and a series of beeping. 53
f) Arrows and beeping (or other audio/voice indicators) will change in size, color, frequency, volume, as the camera is re-oriented—to help indicate how the alignment is improving or worsening, until placement is good 55
g) If the camera moves out of place 54, the auto align process may automatically restart to guide the patients to re-orient camera again. This process can continue until the proper visual is provided.
h) The camera can take a picture of the image when the optimal viewing is achieved 56
d) The image can be texted to the provider during the visit in a format so that contrast can be adjusted to further enhance optimal viewing
i) The image is automatically stored in the patient file so it can be used as a comparison during repeat visits.
d) When a stored image exists for an area of interest, the auto-align feature can be used to optimize the same view with the same lighting to enhance the comparison and change over time. An example would be when an arm has skin infection. Having the same view at the same distance with the same lighting would allow the provider to compare the size, color and shape of the infection from one visit to the next.
5.) Provider determines she no longer needs a look at that specific body part and turns off auto align 57.
6.) Patient can now freely move camera/device again and will not hear/see any alignment notifications;
7.) Provider may choose to initiate auto-alignment process again.
8.) The patient and provider determine the visit is complete 28.
9.) The patient or provider ends the visit/call. This might be done by clicking on “done” or by saying “end call” or the equivalent.
10.) The provider completes his documentation and closes the encounter.

The power of the autoalign is both in enabling the provider to control or provide instructions to the patient to control the phone to align the phone in an orientation necessary, but also in the reproducibility of these images. This allows for consistent placement of a phone for capture of similar images, for example for detecting or visualizing strep throat, the same general angle position can generally identify the same features across one patient or across many patients.

Accordingly, a preferred embodiment is directed towards a system comprising the above components, wherein said components, running an applicable software program, can enable control between the control panel and one or more devices to engage one or more devices to a predetermined position.

A method, is contemplated for capturing an image using the above system, wherein the method steps comprising engaging a mobile device to the system through an application; engaging the control panel to the mobile device; determining a predetermined position for the mobile device; generating an audio or visual signal from said mobile device to orient the mobile device into the predetermined position; generating an audio or visual signal when said predetermined orientation is met; capturing an image at said predetermined position; uploading said image to said system.

Real-Time Feedback

During the call, the system is capable of providing real time feedback to the medical professional on the status and quality of the call. This in turn is intended to ensure a consistent doctor relationship. The video system tracks voice intonations and visual cues to ensure that the doctor is being professional. Furthermore, the video can check the background for ambient light, etc. The goal is to provide a feedback loop, wherein if something goes wrong with the appearance of a professional shoot for the doctor, an alarm will ding to help the doctor correct these issues in real-time.

Indeed, a difficult aspect of performing medical assessment in a voice or video call is to maintain the professional nature of the appointment and to ensure consistent and controlled actions and responses from the clinician. In certain embodiments, the system may utilize software that provides for a real-time feedback and assessment system that monitors several factors for an attending clinician or practitioner. The factors include, but are not limited to: physical appearance/professional attire, eye contact, lighting and image analysis, voice tone, and background noise. These features are important to providing high quality and professional care in telehealth.

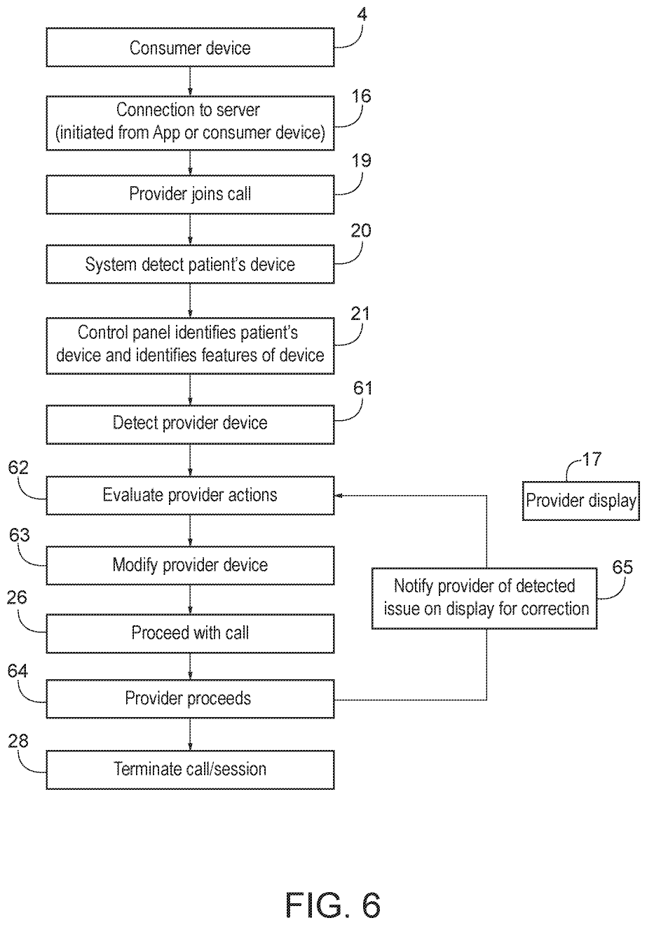

FIG. 6 details a feedback component for the provider. For example, professional attire can be determined by providing a recommended appearance for the doctor and can be compared to a pre-determined approved attire. The clinician and/or the system can use a camera to provide an image of the view the patient sees, to review the attire and appearance before a call starts. Eye contact can be monitored to ensure that the clinician is speaking to the patient, not around the patient or to the table. Background lighting can make the call appear professional and make it so the clinician is easy to see—for example, harsh glare in the image may be distracting. Voice tone can be monitored to ensure that appropriate tone is provided for patient interaction. Background noise can be automatically filtered out, in some embodiments.

Preferred embodiment provide for an optimal setting or input for the various features described above and some alarm or notification system is provided when any input strays from the optimal setting, i.e. background lighting is changed, or voice tone changes. The clinician is then notified that an input is out of optimal parameters and can modify the setting or behavior.

This feedback system thus seeks to improve the appearance and professional nature of the telehealth call by monitoring and providing mechanisms to correct or modify behavior or inputs in real time. Providing consistent, high quality care, in a professional setting allows for telehealth to have greater success and to provide for high quality care in this type of clinical system.

Therefore, real-time Clinician Feedback can further be described as elements in the system to ensure that the patient has a positive experience; which is oftentimes as important as ensuring the clinician can successfully deliver care. In this invention, the clinician is offered automatic feedback about his or her appearance and performance during the visit. The feedback can range from reminding the clinician to focus on the patient (based on tracking clinician's pupil/eye position and focus) to noticing the tone of the voice of the clinician, to commenting on the attire and general look of the clinician. The system here works as follows. Possibly even prior to answering a call, the system mirrors the physician via video and voice so she/he can see herself. The system at this time may automatically evaluate the physician and their environment including background image, lighting, and noise. Also physician state of dress is too much skin showing, is a tie being worn, etc. Recommendations to improve the physicians' appearance will be shared if applicable. A photo of the physician may be captured for quality control and audit. During the patient video visit, the system is constantly reviewing the same metrics as before—but also monitoring physician eye contract, voice tone, and background noise. The system will alert—through the clinician's console if she/he is deviating from the recommended standards of eye contact, tone, etc. The system may also attempt to automatically remove background noise if possible.

FIG. 6 gives further details of the real time feedback.
1.) Patient initiates telehealth visit
2.) Patient determines he has a medical need now or in the future
3.) Patient downloads an app or other electronic solution for remote medical video visits.
   a) The patient creates an account to record medical information for themself and their family and dependents and enable login to system/app
   b) The patient determines that they have a medical need now and they login to the system/app
   c) NOTE—The system/app includes a call center of providers available for calls immediately or at a scheduled time in the future. (the providers may be logged into the system already at this point)
   d) The app connects to a server which notifies a provider that a new call/request/visit needs to be serviced
4.) Provider joins the telehealth visit
   a) The provider accepts the call
   b) The two way communication session between the patient and provider is initiated
   c) The system automatically evaluates the provider based in his video and audio stream
   d) Feedback for corrective action is suggested to the provider based on his behavior, appearance, environment, etc.
   e) Feedback for corrective action is delivered to the provider via his video terminal

### Examples

i. The system determines the audio is not loud enough and asks the provider to speak louder or automatically adjusts the speaker/mic
ii. The provider is moving around too much and asked to sit still.
iii. The provider's environment is not suitable for a professional setting (Examples might be poor lighting, a busy background, or noisy background)
iv. The system notifies the provider to make corrective action or asks the provider to move to a new setting
v. The system determines a provider is not making eye contact with the patient
vi. The system reminds the provider that he adjust his position to improve eye contact and then notifies the provider when sufficient eye contact is made
vii. The system determines the tone of the provider's voice is too harsh
viii. System reminds the provider about best practices for speaking with patients. An example would be having feedback via a color code right beneath the webcam so that the provider adjust his voice into the optimal range. For example, a set of feedback bars may be present to indicate good tone or poor tone, or other issues with sound quality.
5.) the Patient and Provider Determine the Visit is Complete.
   a) The patient or provider ends the visit/call. This might be done by clicking on “done” or by saying “end call” or the equivalent.
6.) The provider completes his documentation and closes the encounter.

One mechanism to ensure quality control is that on the provider display 17 or on the provider's device 5, a teleprompter can be utilized, with the camera for the video feed above that feature, with information of the patient, i.e. EMR, or other relevant information displayed, so the provider is always appearing to look at the patient.

One embodiment, therefore, is directed towards a system comprising said above features, wherein the provider device 5 utilizes its camera, audio and visual settings, in conjunction with the control panel 3, to identify non-optimized settings, or non-optimized actions of the provider; wherein a message is generated to the provider and displayed on the control panel; and wherein the control panel, having engaged the provider device 5, automatically optimizes settings to a predetermined optimized setting, or provides instructions for manual optimization of the same.

A method of generating a consistent and professional telehealth setting comprising; optimizing parameters of a provider device, said provider device being a mobile computing device capable of audio and video communication and said mobile computing device connected to a control panel; said control panel capable of automatically modifying the hardware or software of said mobile computing device; identifying a non-optimized feature in a telehealth setting being generated by said mobile computing device; notifying the provider of the non-optimized setting; manually or automatically modifying the non-optimized setting.

Video Overlay

In performing remote video analysis, having information about the patient, their location, and local data may be helpful in determining a particular ailment, if not otherwise obvious from the symptoms. For example, providing relevant and timely information about a patient to the clinician is crucial. In the case of telehealth, there is an opportunity to overlay such information on the video stream to give it extra attention for the clinician. Some such information may include (based on geolocation and medical condition) public health information−such as recent outbreaks of disease. Video overlay of common questions to ask the patient for given conditions may also be used. These can be provided on the display 17 or the provider device 5, as indicated to ensure quality control in the feedback systems.

Video overlay may include the following features after the call has been initiated. The two way communication session between the patient and provider is initiated. The system searches the World Wide Web clinically relevant medical information related to weather conditions, social determinants of health, address, zip code, geolocation of the patient and other factors that may impact the health of the patient. An example might be pollen counts for asthmatics or weather conditions based that can be combined with the medical history of the patient (as documented in the app or an accessible medical record) such as hot temperature in the elderly who are on medications that might make them prone to heat stroke. Details of these searches, such as the weather (specifically any deviation from normal) is overlaid on the video stream displayed to the provider. Provider views this information (such as high pollen count) and uses it to further his or her diagnosis or treatment of the patient

Video overlay information is determined by using contextual information relating to the patient and searching or matching that data against other systems. These other systems may be public or private databases of information about the patient or a given location, time, or combination of both. The information viewed gets documented within the medical record 12 of the patient.

A preferred embodiment is directed to a system utilizing the above features to engage a video overlay to improve the ability to provide information to a provider. In particular said system generates data from the location of a patient device 4, and collects data regarding current medical issues, e.g. flu outbreak in the area, pollen counts, weather, smoke, etc., to aid in characterization of or diagnosing the patient.

A method of utilizing local environmental conditions in diagnosing a patient in a remote telehealth setting comprising; engaging a telehealth session between a patient and a provider; generating a list of environmental conditions relevant to a diagnosis based on the present and/or past locations of the patient device 4; making a diagnosis of the patient comprising the environmental conditions.

Advanced Visualization

As telehealth technology progresses and we move to even more advanced remote visualization technology, use of two or more cameras, cameras with different filters or processors, to detail IR, visual light, and other spectrums, wherein the combined one or more cameras can be utilized to generate a holographic representation of a patient. This may also be completed by having the patient come to a processing center, upon creating an account, wherein a visualization and representation of the patient is created. This can then be displayed, and modified, by use of the cameras from the patient device 4. In such a case, using tools such as a stethoscope might seem helpful, but are not technologically compatible—or needed. However there will be a learning curve as clinicians being able to treat visually via a hologram (and inside the hologram) vs. listening to what is happening inside is quite a different art. The microphone from the user device, the accelerometer, a heart rate monitor, or other feature can then be used in conjunction with the holographic image to show or recreate the sounds (of heart beating, or lungs exhaling) based on video of patient. This puts the information needed for diagnosis back into a format that the clinician is used to.

At the same time, the provider can then detect sounds from the patient and generate those to the hologram where a visualization of the possible or probable cause can be identified. For example, lungs may be shown with excess fluid, the trachea with a growth, the nasal passages inflamed due to illness or allergy, or the heart having a blockage. The combined visual and audio files can therefore allow such unique representation and remote diagnosis.

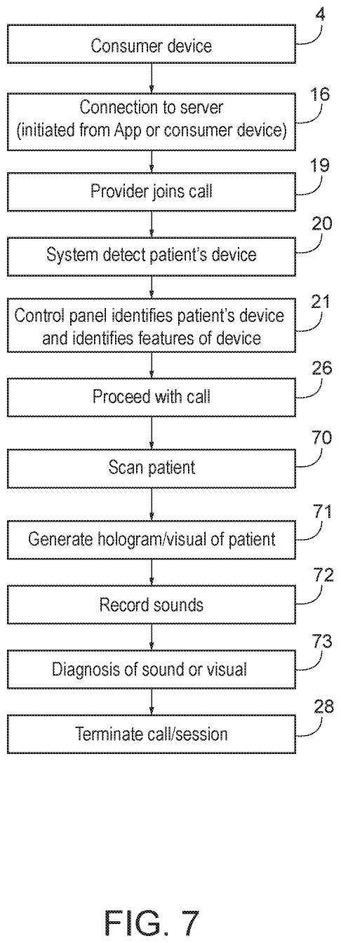

FIG. 7 details the ability to scan a patient and to generate a hologram of the patient, which can be used, in connection with other features of the phone, i.e. listening to sounds from the patient, to improve remote diagnosis. FIG. 7 specifically details:
1) Patient initiates telehealth visit 16 with their device 4, and the device is detected by the system 20 to identify features of the device for control 21.
2) Provider joins the telehealth visit 19
   a) The provider accepts the call
   b) The two way communication session between the patient and provider is initiated 26
3) The provider wants to hear how air is moving through the body of the patient. An example might be “listening” to the airflow through the lungs or bowel sounds in the gastrointestinal track
4) The system which is scanning the patient 70 in real time to record the visual, spatial, and volumetric information about the patient analyzes the movement of the body parts inside and outside the patient (the system is using XRAY and other technologies to scan the inside of the patient as well).
5) The system recreates the geometry of the patient's anatomy, waveflow of the air and simulates the sound caused by airflow through the body 71.
6) The system records and plays the sound 72 via audio to the provider.
7) The provider is able to use the sound information for diagnosis or treatment of the patient 73.
8) The patient and provider determine the visit is complete 28 and the provider completes his documentation and closes the encounter by updating the EMR 30.

A system as provided above, capable of utilizing sound and video inputs to generate a holographic image of a patient; utilizing said sound and image inputs to generate a holographic representation of movement of the patient based on said inputs.

A method of diagnosing a patient from a hologram comprising: generating a holographic image of a patient comprising one or more sound and image inputs to generate a holographic representation of a patient; generating a moving holographic representation based upon the sound and visual inputs; determining a diagnosis from said holographic representation.

Emergency Action Plan and Sending Assistance

In certain instances, a call may proceed where, it becomes clear that the patient needs immediate medical attention. For example, the patient has initiated the call, complaining of chest pains, and the medical practitioner suspects that the patient is, or has had a stroke or heart attack. For example, a patient may complain of abdominal pain and the medical practitioner may suspect that there is a ruptured spleen that may go septic without immediate attention. Regardless, there are any number of instances and reasons why a patient might need immediate medical attention. Accordingly, within the system, the medical provider is able to identify the location of the patient and send medical personnel to that location. For example an ambulance can be sent directly to the patient to take them to the hospital, urgent care, or other medical center for immediate attention.

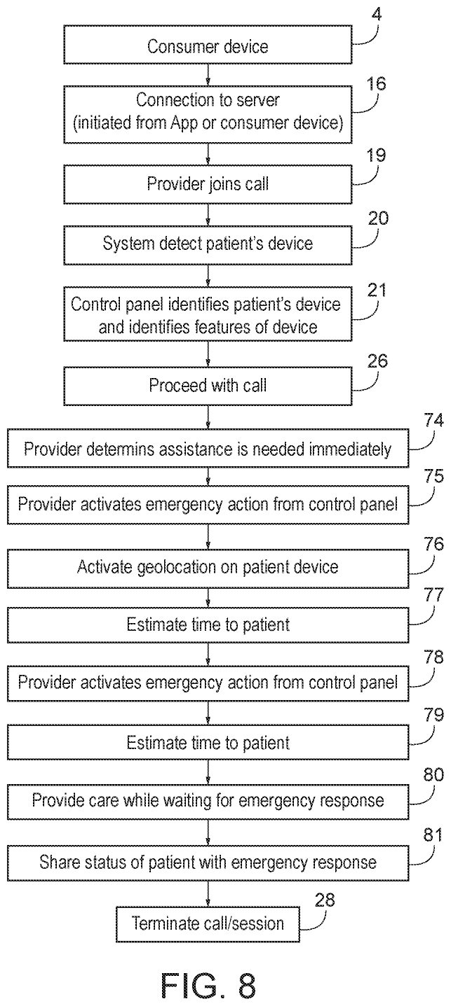

FIG. 8 details a situation where immediate assistance is provided to a patient.

Patient initiates telehealth visit 16 with their device 4

a. The app connects to a server which notifies a provider that a new call/request/visit needs to be serviced 16. The patient's device is identified 20 and the control panel 3 identifies features of the device 21.

Provider joins the telehealth visit 19 and accepts the call
a. The two way communication session between the patient and provider is initiated 21.
b. The provider determines the patient will require transport to a medical facility to complete treatment or diagnosis 74.
c. The provider uses his terminal to select ambulance dispatch 75.
d. The patient's phone shares its geolocation data to the system 76.
e. The system estimates the duration of travel for pickup of the patient based on distance and traffic and determines which ambulance service will be the fastest responder 77.
f. The system notifies the first responder (ambulance) of the patients name, location, and medical problem 78.
g. The ambulance is dispatched to the correct location and a further estimation of time to the patient is provided 79. This estimation is provided continuously, with the dispatch.
h. The video visit provider cares for the patient and provides treatment while the ambulance is traveling to the patient 80.
i. The ambulance arrives, assumes care of the patient and the patient is transported to the hospital or alternate care facility.
j. The video visit provider shares current status of patient with EMTs 81.
k. The video visit provider may remain on the call and as part of the care team while the patient is in transit to the facility.
l. The provider ends the visit/call 28 and the provider completes his documentation and closes the encounter 30.

The above figures and features provide certain actions to improve the quality of the telehealth session, or to perform certain actions to allow for improved visualization or diagnosis in such a session. However, we can also address the telehealth call as improving the workflow, to enable seamless processing from initiation of the call to treatment and more.

Indeed, systems require that we target and improve pre-call and post-call experiences for a consumer. For example, a patient who is going to undergo a first telehealth appointment may receive a pre-call email, a call, a video chat with a nurse, or a recording, to go through the procedures. Other information may address any further concerns that the patient may have, in order to provide further information for the provider, before the call.

Indeed, telehealth appointments may be scheduled in advance, thus identifying a particular doctor at a particular time. Other telehealth sessions may be started simply at the request of the patient, who feels the need to receive treatment or diagnosis of an immediate medical need. Both scheduled and individual on-off visits are necessary for patient care, and thus we consider both to be essential for providing for medical treatment.

Thus, an embodiment is directed to a method of sending emergency care to a patient in need thereof comprising: generating a telehealth session with a patient; determining the need for an emergency interaction; engaging a control panel to identify the location of the patient; sending emergency care professionals to the then present location of the patient; providing instructions to the emergency care professionals of the status of the patient.

One Click Doctor Visit

As soon as the app is launched, an intake coordinator, or clinician is available via video. This reduces any barriers to the patient calling/connecting. They don't need to remember passwords, accounts, or other information. In some cases an intake specialist will collect name/insurance/allergies, etc. from the patient and then do a warm handoff to clinician. In some cases the one click process will start after the login.

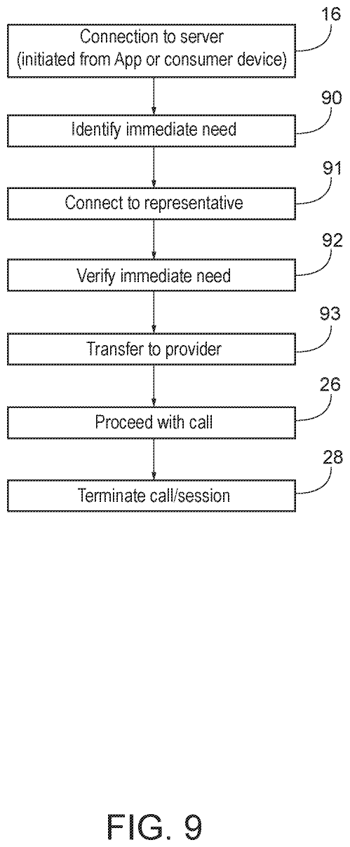

FIG. 9 details a flowchart of a one-click telehealth visit.
1) Patient initiates telehealth visit
   a. The patient determines that they have a medical need now 90 and they access the system/app 16. The system/app includes a call center of providers available for calls immediately or at a scheduled time in the future. (the providers may be logged into the system already at this point)
   b. The app connects to a server which notifies a provider that a new call/request/visit needs to be serviced
2) The app immediately starts a video session with a representative from the health system 91. The patient is not asked to login or enter any information, once the app is initiated the next thing that happens is the patient having a video discussion with a representative for the provider (or the provider himself). As an example, it is similar to activating an app that remembers the login information and does not require the user to login with each use.
3) The representative asks the patients a number of questions to verify the identity, reason for call, and payment information 92. By use of the App, some of this information is automatically collected, and can be generated from the provider end without need to verify the same.
4) The representative enters all of this information into the system and then transfers the patient to a provider to handle the call 93
5) Provider joins the telehealth visit and the two way communication session between the patient and provider is initiated 26
6) Provider dispenses medical care
7) The patient and provider determine the visit is complete 28 and the patient or provider ends the visit/call and the provider completes his documentation and closes the encounter.

This workflow enables a patient to quickly connect to a provider and to get guidance or assessment of a medical need, without the need to schedule an appointment. Take a regular consumer, who has a fever for a day or two, they are required to either call and schedule an appointment, or to go to urgent care or the emergency room. There, they wait for an undefined amount of time, possibly spreading illness to others and only then getting care. The above one-click, allows for fast response, at home, with discussion with a provider who can then guide the patient on whether a visit to urgent care or emergency is needed. If such care is needed, the provider can recommend a particular location, send the patient information to that location, which can prepare a space for the patient before their arrival. This facilitates the flow of the patient from home to hospital setting, if such hospital setting is necessary.

An embodiment is directed to a system having the above features and for providing for one-click access between a patient device 4 and a telehealth professional.

A further embodiment is directed to a method of generating a professional medical connection between a patient and a professional with one click; engaging an application on a mobile device; identifying a medical need on said application; connecting said mobile device to a health care provider; confirming the medical need from said application with said patient; and diagnosing said patient based upon communication between said provider and said patient.

Warm Handoffs. Warm handoffs have become increasingly common with voice phone calls in call centers. Expanding this concept to telehealth and providing warm handoffs in the case of video calls is even more important. Discussing health is extremely personal, and it is already challenging to shift that discussion to electronic mediums. By providing warm handoffs between practitioners, the patient is afforded extra comfort with the technology that is imperative when delivering healthcare via this medium.

The workflow here is likely that (1) a patient calls an 1800 number call center asking for an appointment. (2) The operator offers an in person appointment in a couple days, or a video visit immediately. (3) If the patient chooses video visit immediately, the operator initiates a process whereby the patient's phone is activated (get a notification) to start a video call or if they don't have our app, they get a text telling them how to download the app, and start the call. (4) the operator also picks the appropriate physician and sends the patient context and problem to the physician. (5) the patient disconnects from the voice call and completes a video visit. (6) At the completion of the video visit, the patient is offered to schedule a follow up online or offered to be routed to the call center to schedule another appointment. (7) The routing to the call center happens right within the app, such that the patient does not need to leave the app and start another call.

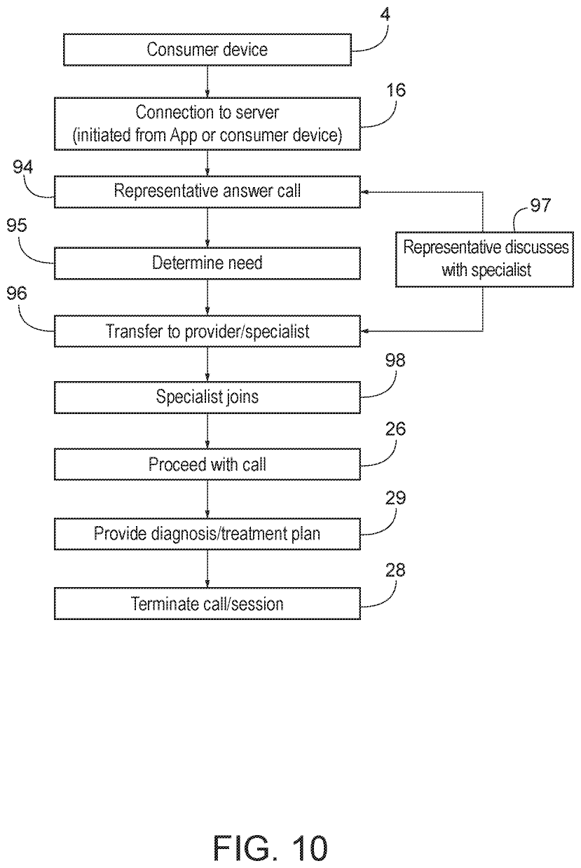

FIG. 10 provides a detail of a prospective warm handoff. Omitted are certain aspects of engaging the call, as provided in FIG. 4. A patient initiates telehealth visit through their device 4 and connects to the server 16. A provider or representative answers the call 94 and a need is determined 95. The two way communication session between the patient and provider is initiated and the initial person who take the call (whether a provider or non-provider staff member) determines that another person is required for to render further care 95. If the initial person is staff, the hand off may be to a provider. If the initial person is a provider, the hand off may be to a specialist or another provider 96. Optionally, the initial contact may discuss the matter with the provider or specialist 97 before the transfer to the patient is complete. There is no limitation on the number of transfers that can be done within a single call, as the particular specialist may be determined through subsequent evaluations.

Accordingly, as the call proceeds, the provider at the moment, may select an available and appropriate second provider (for example, a specialist) from a list of available specialists on his screen. The provider has the ability to share the photos and biographies of the specialists with the patient (on the patient's screen) or on the provider's screen. The specialist is joined into the call, as a 3-way video call 98. The initial staff member or provider introduces the specialist to the patient and the call proceeds 26. The initial staff member or provider has the ability to leave the call or to remain, as necessary. For example if the initial member is the patient's primary care physician, she may want to remain on the call to better understand the diagnosis and follow-up treatment necessary for the patient. The providers give a diagnosis or treatment plan to the patient 29 and the patient and provider(s) determine the visit is complete 28. The initial and second provider complete documentation and the encounter is closed by the last provider to complete documentation. The system ensures that documentation is completed by all providers.

A method of transitioning a call between a first and second provider; comprising engaging an application on a patient device; generating a connection between said patient device and a telehealth system of the present disclosure; connecting said patient device to a provider; connecting said patient device to a second provider automatically.

Convert Voice to Video and Video to Voice

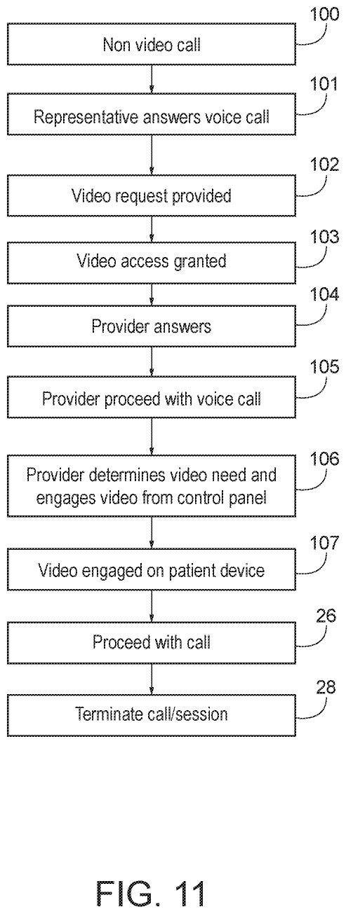

FIG. 11 details the issue of converting from a voice call to a video call, or the reverse, as necessary for treatment. The ability to convert a standard phone call from voice only to video is a new advantage in the world of telehealth. Right now, the process to initiate a telehealth call is fairly cumbersome. A simple button press during a regular voice call, which initiates a video call is a great step is removing barriers to this technology. There are numerous ways that technology can facilitate this idea. In the simplest example, while on a voice call and a patient is trying to schedule an appointment, they could be asked to press (a number) to initiate a video call with a provider instantly. Upon pressing (a number) they would see an incoming video call and be able to start speaking to that provider immediately. That provider will also be able to access all the context which was shared during the audio only portion of the call. In other examples, while discussing a concern with a clinician or provider via a standard phone call, the provider could switch the call to video to enhance the discussion and care provided. The reverse of this process is also important.

Convert voice to video as depicted in FIG. 11.
1) A patient seeks medical care by voice (non-video) calling the health system via a phone or mobile phone 100.
2) A staff member at the health system determines what type of care is needed 101.
3) The staff member asks the patient if she would be amenable to an immediate video visit 102
4) The patient confirms her interest in such a visit 103. The staff member asks the patients a number of questions to verify the identity, reason for call, and payment information. The staff member uses his system to determine what providers are currently available
5) The staff member selects a provider who would be appropriate for the patient and a provider answers 104 and proceeds with the voice call 105. The provider, recognizing the need to video analyses 106 engages video from the control panel 3.
6) The system engages the patient's phone to activate video, which is engaged 107 automatically or through accepting a request. For example, a request may use the patient's phone number, email or telehealth app to automatically send a command to initiate a video visit to the patient, or it may be a click on the patient's device to simply accept the video feed. This can be achieved through an email or text message link, or directly to an app previously installed
7) The call proceeds with video 26 and the session is terminated upon completion 28.

The purpose of this feature is to allow for access and to activate that access from voice only to video, thus giving the practitioner the ability to see and examine the patient. At the same time, it may be necessary to end the video feed during a call. For example, the examination requires visualization of the genitals of the patient, and after review of the area, the video feed can be terminated to protect the privacy of the patient, and to prevent the feeling of discomfort on behalf of the patient. Thus, the call can proceed with voice only, after review of the relevant issue.

A method of generating a video call from a voice call comprising: enacting a voice call between a patient device and a provider; detecting the parameters of a patient device; requesting permission for a video call; receiving permission for a video call; determining a need for a video call; activating a video call between a patient device and a provider device by generating a video call from a control panel; connecting the video call between the patient device and the provider device.

Transferring Calls/Patients

A significant challenge in telehealth today is that licensing restrictions require providers to be licensed in the same physical location of the patient. This restriction poses a challenge for telehealth systems, as the patients can easily call from any location. The invention here allows that patients can be handed off (via hard or cold hand off) to partner telehealth centers which may be on different or separate telehealth platforms or solutions, based on the needs of the patient. A patient in Florida might reach a clinician via telehealth which is only licensed in New York. This clinician would then have an easy workflows to hand that patient off to a clinician which is licensed in Florida. Additional, handoffs could take place to facilitate matching specialists, preventing conflicts of interest, or other reasons. The handoff process would allow sharing of medical records and referral fees where appropriate.

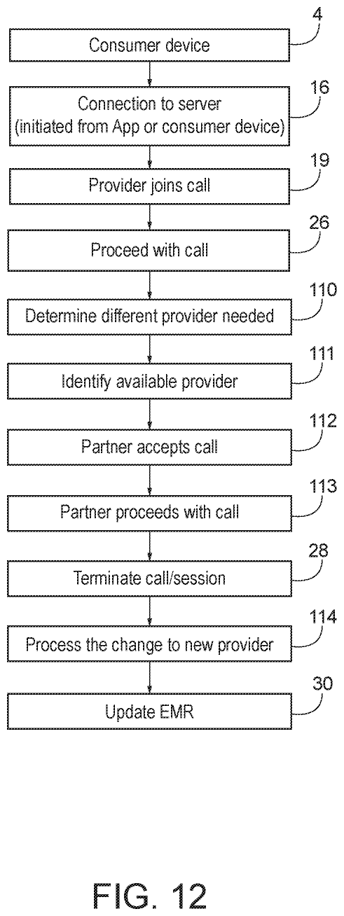

FIG. 12 details a handoff process as necessary to meet such needs.

Patient initiates telehealth visit as provided in part as by FIG. 4. The app connects to a server 16 which notifies a provider that a new call/request/visit needs to be serviced. The provider joins a call 19 and proceeds with the call. The system or individual provider determines that based upon a specific need that the patients should be treated by a different provider 110. This can be based upon items such as volume of calls in the queue, time delays on part of the originally designated provider, specialty, licensing restrictions or geographic location the patient. The System identifies another provider 111 available within the same health system. If none available, it identifies a provider within a partner network. Algorithms can determine which partner network based on best match (including location, quality performance metrics, service metrics, past patient satisfaction and financial value). The Partner network accepts call request (this can be programmed to automatic or require approval). Technology—back end processing records and handles the payment and logging/auditing of the call (between the initial call network and the recipient network). The Provider in secondary network joins the telehealth visit 112. The two way communication session between the patient and provider is initiated 113. The provider treats or diagnoses the patient 29. The patient and provider determine the visit is complete 28. The provider completes his documentation, updates the EMR 30, and closes the encounter.

One aspect of this transfer requirement, is that the particular need to transfer can be really limitless. For example, the provider is not the proper specialist, the provider is too busy, the patient needs in-person care, and another network is closer, the patient prefers a different gender caregiver instead of the current provider, based on comfort with a gender specific issue. The ability to find a provider, access that provider, and seamlessly get that provider on the call ensures that the patient does not have to end one call, find a number for another provider and wait for that call, etc. This gives seamless access between professionals and the patient.

Discharge Information and Follow-on Care

Perhaps the most important part of any patient and clinician interaction is the follow up care. Telehealth lends itself well to having discrete calls, but does not necessarily prompt that patient to seek that follow up care that they should. Furthermore, certain ailments require specific care procedures to ensure that the treatment is correct.

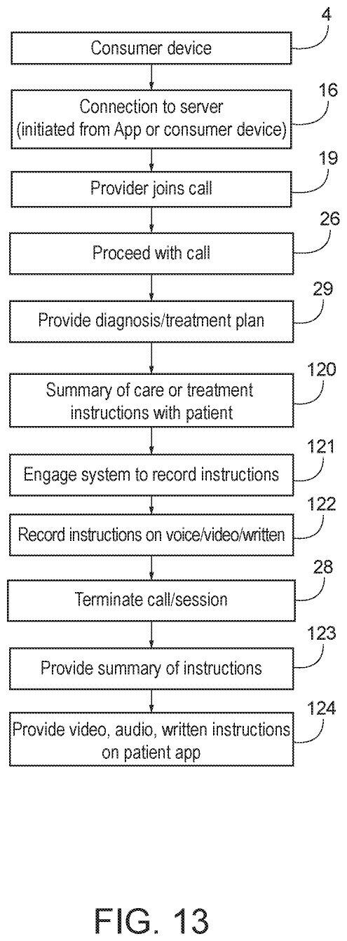

FIG. 13 details a process within the system for Recording discharge instruction, which can then be accessed by the patient at any point, to help with treatment protocols. Indeed, sending a discharge instruction packet is an important part of any clinical visit. However, having the specific words spoken by the clinician available for replay is an additional benefit. In this embodiment, the patient and clinician can jointly opt in (at the end of the visit) to have the visit summary discussion recorded (either video or just audio) and available for replay at a later time. This recoding may also only be available for X period of time.

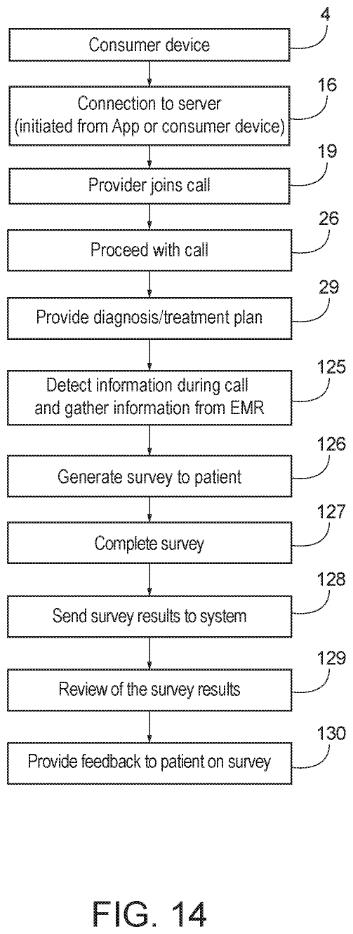

1) FIG. 14 shows the initial features of the process, connecting to the server/telehealth 16 with a consumer device 4, wherein the provider joins the call 19 and the call proceeds 16. At some point, the provider provides a diagnosis and/or treatment plan 29 and the Provider determines he is ready to share a summary with the patient 120. The provider selects that he is ready to end the call and start summary and engages the system to record the instructions 121. The System begins recording video session 122. These spoken words can also be recorded and created into a text or written profile, or simply available as an audio file. Furthermore, the instructions can be translated, if necessary for a patient or caregiver to have the instructions in a different language. The patient or provider ends the visit/call 28. The summary of instructions 123 is then available on the application of the patient device, or from the patient's file. A written summary of the visit is sent to (or available via portal) the patient. The summary includes the audio/video of portion of the call where the summary instructions were given. The patient can watch and re-watch this section of the video for a reminder of their next steps on the application 124.

Further steps can also be included, for example: FAQ with regard to the particular drug, treatment, disease. A set of things to do and not do, for the patient, and information regarding medication, exercise, diet, etc. that may be helpful for treatment. Additional materials from Videos and pre-existing video or links can be added from websites, computers, tablets or smart phones. An example might be for a patient who has a sprain. The provider might give instructions via audio/video but then can also add a link to a video with specific range of motion exercises.

A method of generating a recording of a voice or video discharge information to a patient using a mobile application; generating a telehealth session between a patient device and a telehealth system, said system comprising a central hub, a control panel; engaging to a provider device; facilitating communication between the provider device and the patient device; engaging the control panel to record discharge information; transcribing the video instructions to a written transcription; sending the patient a video recording and written transcription of said discharge information to said mobile application on said patient device.

Dynamic Surveys

At the conclusion of a telehealth session, the patient may be engaged to stay on the line for a post-call survey. This may be achieved simply by requesting feedback, or providing some incentive, for example providing a coupon or other code to reduce the cost of care or of a medication. A survey can also be automatically generated and sent to the patient, for example to their App, where completion can be again requested or an incentive provided. This will help provide information regarding the quality of the services provided and the level of care and satisfaction for the particular visit. This information is crucial to optimizing the care delivered through this medium. Different patients (based on geographical location or severity and type of medical condition) might need to be asked different follow up survey questions. A patient local to our facility might be asked about follow-up care in our facility, whereas a patient not geographically proximal to our location may not.

FIG. 14 provides a detail of generating and delivering a survey. The patient engages the system through their device 4, and connects to the server 16. The provider joins the call 19 and the call proceeds 26. Ultimately, a diagnosis/treatment plan is generated 29.

A survey is automatically generated by the system by detecting information 125 from the call and gathering information from the EMR. For example, during the call, the system detects key words and uses these key words to identify issues being faced. This may provide a suitable set of information regarding the care. For example, a call discusses cataracts, and the survey identifies that cataracts are related to eye issues and generates questions from the survey on the eyes. Or, the call discusses irregular bleeding due to a new contraceptive device, and the system generates surveys tailored to such discrete issues, that may further include questions regarding preferred provider gender, as may be requested by a patient. Then, the system automatically generates a survey for the patient 126. The survey is dynamically created for each patient.

Survey questions may be tailored based on any or all of the following parameters:
a. Demographics such age, gender
b. Past medical history in the chart of the patient
c. Reason for call
d. Key words or phrases mentioned during the call (for example, medical conditions, access difficulties, reasons for using telemedicine)
e. Time of day
f. Location
g. Prior visit history
h. Discharge instructions (for example, did they need an antibiotic or a splint)
i. Compliance with discharge instructions (getting medications or making follow up appointment)
j. Alternative options for care for the medical problem being evaluated in this visit.
k. Provider which delivered care quality of call
l. Technological challenges during call.
   i. Example: A patient located near a medical facility would be asked about intent to seek follow up care at that specific nearby medical facility
   ii. Example: A patient who's call had poor quality (as detected by the system) might be asked more detailed questions about call quality

The patient can receive the survey by anyone of the following means:

a. Immediately shown the survey at the conclusion of the call

b. The survey can be sent within or outside the app at a designated time point following the call (for example, one might want to wait 10 days to assess whether the patient received his follow appointment, but you might want to find out if the patient received their medications 1 day later)

The patient completes the survey 127 and the results are sent to be reviewed by providers 128. The survey results can be saved for review by system administrators 129. The system will enable programming so that some survey results can be forwarded to providers or staff to facilitate intervention (for example, when patients have not yet gotten medications). Finally, feedback may be provided to the patient or provider based upon the survey 130.

In a preferred embodiment, a method of generating survey data and generating a survey comprising; engaging a telehealth session between a patent device and a provider device, through a telehealth system comprising a control panel and a server; detecting key words or phrase during the telehealth session and pulling data from the EMR records to identify survey information; generating a survey based upon the EMR and key words or phrases.

Targeted Coupon Codes

Getting patients to seek the care they need is extremely challenging. Offering incentives, via coupon codes (or other discounts) is one tool which can be used to incent patients to get such care. In many cases, offering discounted, or free follow up or preventive care is less expensive than offering nothing and waiting for a medical problem. In this invention, discounts are targeted to patient based on their geolocation, medical conditions, and time since last visit. A patient with a chronic condition who has not been seen in two years may be given more of a discount (even to the point of given a positive reward) for completing a telehealth visit whereas a similar patient who has been seen within the year might only be given/offered a smaller discount/incentive to participate in a call. Furthermore, coupons may be given for answering surveys or for providing additional feedback or information on the App, as is appropriate.

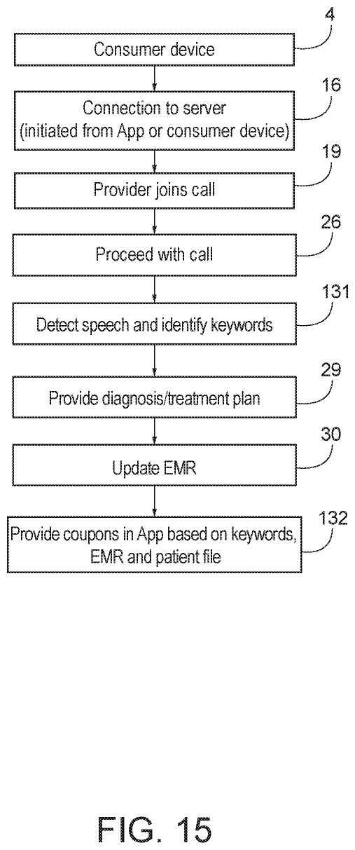

FIG. 15 details a brief overview of identifying coupons of interest and providing the same to the patient. The Telehealth call is generated by a user device 4 and connects to the server 16. A provider joins the call 19 and the telehealth session proceeds 26. During the call, as with the dynamic surveys, the system can detect and record key phrases or words, which are related to a set of offers that may be available. A diagnosis/treatment plan is provided 29 and the EMR is updated 30.

For example the system, in a database, contains a set of coupons that have been offered by manufacturers, or by the health-care system, or by the insurance provider, etc. These coupons can be coded to meet certain key words or diseases or treatment options. The EMR is the first location for referencing, where anything on the EMR can be cross-reference to the database of coupons to identify a match. Furthermore, key words provided during the call can be utilized in conjunction with the EMR. Results should be time oriented, thus recent key words or entries in the EMR should have a higher priority, i.e. they are current issues, than for older terms, that may now be irrelevant. For example an EMR has a record from 3 years ago for a broken leg, and a current call for a bacterial infection. The broken leg entry is likely to be so old that no consumer would need coupons or products related to that broken leg, and thus the coupons should be targeted to the bacterial infection issues.

After completion of the telehealth session, and use of the terms and EMR information, coupons can be generated to the patient. The system automatically generates a coupon code or incentive for the patient for follow up care, either through additional telehealth visit or in person visit. The coupon code is dynamically created for each patient. The coupon codes can further include coupons based on a visit or the lack of a recent visit.

The amount of the code and other details of it are automatically determined by the following:
a. Time between last visit and current date (Note: in this case steps 1-6 may not have occurred recently)
b. Reason for visit
c. Key words or phrases mentioned during the call (for example, medical conditions, access difficulties, reasons for using telemedicine)
d. Time of day
e. Location
f. Prior visit history
g. Discharge instructions (for example, did they need an antibiotic or a splint)
h. Compliance with discharge instructions (getting medications or making follow up appointment)
i. Alternative options for care for the medical problem being evaluated in this visit.
j. Provider
k. Poor technological quality of call

For example: A patient who has a chronic condition and has not had a visit within a specified time period would be offered a free visit. Finally, the coupon code or incentive is sent/presented to the patient by email, text message or within the app 132. The coupon code process can be initiated at any point by a health system, not necessarily only as part of a telehealth visit. Coupon codes can be single use or multiple use. They can be restricted by patient, family, health plan, employer or contractor. They can have no expiration date or have a set expiration date. They can be automatically or manually generated and adjusted manually with administrative access

An embodiment therefore is directed towards a method of generating of data from an EMR and from voice recognition systems, wherein said data is utilized for generating coupon codes to a patient comprising: a telehealth system, comprising a database of key words and phrases and an EMR having coded medical information; recognizing spoken words and comparing said spoken words to said database to identify spoken words matching the database; generating a list of spoken words and coded medical information data.

Post Call Scheduling

Perhaps the most important part of any patient and clinician interaction is the follow up care. Telehealth lends itself well to having discrete calls, but does not necessarily prompt that patient to seek that follow up care that they should. Embedding a workflow at the completion of a telehealth call that requires (or pushes very hard) for a patient to schedule a follow up visit it required. This follow up visit could also cause a monetary reward (slight discount in cost of visit) if successfully scheduled. There are a few ways in which the follow up scheduling could happen. At the end of the call, the patient could be shown a list of times for follow up appointments, and the patient could schedule online. The patient could also be transferred automatically to our call center to schedule a follow up via voice. Or a facility might employ telehealth visit coordinators, in which case the patient might be given a warm handoff to someone who schedules their follow-up via video, as outlined above. In other examples—a facility might employ telehealth visit coordinators, in which case the patient might be given a warm handoff to someone who schedules their follow-up via video.

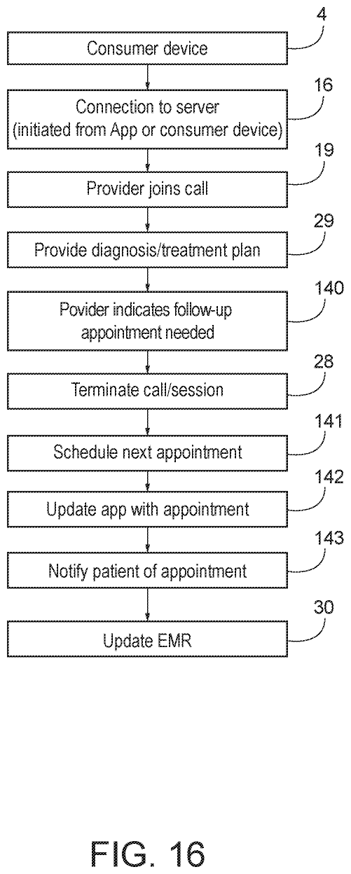

FIG. 16 details a method for providing post-call scheduling through the system.

Patient initiates telehealth visit from their device 4, and connects to the server 16. The provider joins the telehealth visit 19 and the visit commences wherein a communication session between the patient and provider is initiated. The Provider dispenses medical care 29 to the patient and the provider indicates the need for a follow-up appointment. 140.

The patient and provider determine the visit is complete 28. Before exiting the system, the system presents the patient with a list of available follow up appointments 141. This can be immediate within the program or via email, text with linkage back to the scheduling program (As an example, some patients may complete visits without their calendar in front of them so we will provide immediate access to scheduling or can send the patient a link to self-scheduling their next video or an in person visit. The patient chooses a follow up appointment time and provider or the patient chooses to defer the scheduling until they receive an email or text

The appointment is scheduled for follow up or the patient chooses to defer the scheduling until they receive an email or text and the App is updated with the same 142. a. The person receives a notification of an appointment 143, such a reminder emails at predetermined times that they have input into. An example would be a system that automatically sends reminders 1 week and 2 days before the appointment. The patient could also request another on the morning of the appointment.

Therefore, a method is directed towards scheduling a follow-up telehealth session comprising: identifying the need for a follow-up telehealth session; generating a list of available session times; selecting an available session time; generating an appointment time into an application of a patient for said follow-up telehealth session; and generating a reminder for said appointment in said application.

Virtual Waiting Rooms

At a break in the telehealth appointment, there may be a time when the medical professional steps away to put information into the EMR, or to check for prescriptions, etc. The patient will be taken to a virtual waiting room at the click of a button on the control module, similar to putting someone on hold. In a video call, this will provide an advertising or informational opportunity with videos on the user's device. Advertising on the platform is generated to provide the second user (patient) with relevant advertising based upon key words from the telehealth appointment, from the patient's EMR, and from the patient's information generally. In a voice only call, text can be read to the patient or voice only advertisements or information may be played during the duration of the virtual waiting room.

Finally, at the end of the call, the virtual waiting room can again be utilized to conclude the call. Certain embodiment may use a check-out step so as to ensure the patient knows the next steps, such as obtaining a medication, scheduling a follow-up call, or an in-person appointment, etc. Here there will also be a further opportunity for advertisement placement or for information to be presented to the patient.

There are already opportunities to advertise in apps, but in this case we will be advertising throughout the telehealth workflow. Some companies have considered advertising in a telehealth virtual waiting room, but that ideas lacks the true value of being able to target advertisements based on patient conditions. Note: A telehealth virtual waiting room is the time between when you select a doctor and the doctor actually starts the video call with you. This invention includes advertising through scrolling marquees and banners during the patient visit, post the patient visit, throughout follow up emails sent to the patient, and embedded in the discharge instructions delivered to a patient. All such advertisements will have the ability to be targeted for specific patients through data which was obtained during the visit, or during prior visits. Data will be pulled from the EMR, and potentially in real time through voice analysis and natural language processing of the discussion with the provider. Delivery of advertisements for relevant clinical trials can be a major focus of such advertising. As with many advertising related inventions, this invention may start not with providing advertising content, but providing pertinent information or education to the patient in all the forums that may also include advertising if appropriate. Examples are—seeing education about smoking cessation if you are smoker, or seeing information about flu vaccine if it is flu seasons and you have not already gotten it.

These aspects, alone, or combined, can be utilized to improve patient experience during a telehealth call. Indeed, it is particularly suited to use one or more of the aspects together to improve the quality and experience of a telehealth call.

Advertising

In certain embodiments, a patient may be connected to the system but a medical professional is not actively engaged with the patient. For example, this is similar to a brick and mortar facility, where there is a waiting room, after a patient has checked-in. Similarly, there are frequent times when a doctor or nurse sees a patient, but then leaves the room to procure supplies, to check on medications, or other necessary tasks relevant to the patient's appointment. In each case, there is a period of time where the patient is not interacting with a medical professional.

This can also occur during a telehealth call, when the medical professional must step away for any number of reasons. The system can then be engaged to create a virtual waiting room, wherein the patient is provided with voice or video as they wait for additional communication from the medical professional. During this time, there is ample opportunity to provide the patient with directed information related to products that maybe helpful to the patient, educational information, particular videos or recordings related to a medication or illness, or other content that may be relevant to the patient. Therefore, the virtual waiting room may be utilized before the actual doctor patient interaction, during breaks in the appointment, post visit, and through follow up communication sent to the patient, and embedded in the discharge instructions delivered to the patient. The content, can be specifically tailored to the patient based on the data obtained during the telehealth appointment, or during prior visits, or based upon future visits. Data can be gathered from the EMR and also based on real-time evaluation of voice analysis and natural language processing of the discussion with the medical provider.

In particular, delivery of advertisements related to relevant clinical trials can be one form of advertisement. Further advertisements may also include relevant educational materials regarding the issues addressed by the visit or faced by the patient. For example, a patient who smokes may receive smoking cessation information. This may include detailed information, possible medications, other treatment options, and the like. Similarly, if a patient is a child due for vaccines, relevant vaccine information may be provided.

Additional advertising may be present based on a combination of data gathered from the EMR, from the patient information, but also from the words communicated during the call. For example, real-time voice recognition may generate a list of key words utilized during the call and then generate relevant advertisements of information based upon those key words. Advertisements or educational information may also be provided in a list of topics, and the patient can select to hear information on topics. These topics can be populated based upon the list of key words, the EMR, patient records, or a combination thereof.

Therefore the Proposed Invention most likely provides the following general claim limitations relevant for this analysis: (1) A telehealth meeting (2) Identification of relevant Medical information through the EMR and/or through real-time voice analysis, (3) Delivery of targeted advertisements based upon processing of information from (2), (4) Transmission of (3) during the telehealth meeting, in the virtual waiting room, in the follow-up materials, and embedded into discharge materials.

1. A system comprising a central hub, a control panel electronically connected thereto, a first communication device and second communication device; wherein the control panel is enabled to control at least one feature of the first communication device, wherein the central hub generates a voice or video connection between the first and second communication devices, and wherein the control panel automatically optimizes the second communication device.

2. The system of claim 1 further comprising a real time monitoring of the first communication device, said real time monitoring comprising optimizing the parameters of the first communication device, providing feedback to the provider to modify one or more actions, and optimizing parameters of the first communication device.

3. The system of claim 2, wherein the real time monitoring provides feedback on the control panel to automatically or manually modify one or more parameters on the first or second communication device.

4. The system of claim 1 further comprising a video overlay which is displayed on said control panel, said video overlay providing geolocation of the second communication device.

5. The system of claim 1 wherein said central hub is capable of recording voice or video, and said control panel capable for engaging recording status; and recording voice and video of instructions to a patient.

6. The system of claim 5, wherein the recording of voice and video of the instructions to a patient are transcribed from a recording to text.

7. The system of claim 1 wherein the control panel is engaged to control one or more features on the second communication device; wherein the control of one or more features engages with a gyroscope on said second communication device to orient said communication device into a desired position.

8. The system of claim 7, further engaging the accelerometer of the second communication device.

9. The system of claim 7 further engaging at least one camera on said second communication device.

10. The system of claim 7 wherein the second communication device generates sounds or visual cues for orienting the second communication device into said desired position.

11. The system of claim 1, wherein a communication is generated to a third communication device, based upon need or state licensing requirements to provide medical services.

12. The system of claim 1, wherein the central hub is capable of detecting speech and for identifying key words or phrases related to medical care; wherein identification of key words or phrases are combined with an electronic medical record (EMR) having codes corresponding to medical information is accessed by said server, and said key words or phrases and said EMR are utilized to generate a survey.

13. The system of claim 1, wherein the central hub is capable of detecting speech and for identifying key words or phrases related to medical care; wherein identification of key words or phrases are combined with an electronic medical record (EMR) having codes corresponding to medical information is accessed by said server, and wherein a coupon code is generated in response to the key words or phrases and said EMR.

14. The system of claim 13, further comprising a database stored on said central hub of coupon codes, wherein the key words or phrases and the EMR are compared to the database of said coupon codes for identifying coupon codes for deliver to the second communication device.

15. The system of claim 1; wherein the server generates an automatic message to the second communication device for scheduling a secondary call.

16. The system of claim 1; wherein a voice connection is first generated and wherein the control panel is engaged to modify the connection from a voice connection to a video connection; wherein the control panel and server generate a signal to the first communication device and the second communication device to activate a camera and engage video mode.

17. The system of claim 1, wherein the central hub is capable of detecting speech and for identifying key words or phrases related to medical care; wherein identification of key words or phrases are combined with an electronic medical record (EMR) having codes corresponding to medical information, accessed by said server, and said server further comprising a database comprising advertisements corresponding to one or more key words, phrases, or codes in an EMR, and wherein said key words or phrases, or EMR codes are compared to said database comprising advertisements, and wherein said advertisements are delivered to said second communication device.

18. The system of claim 1 or combinations thereof.

19. A telecommunications system for connecting a central hub, a control panel and at least two communication devices; said central hub comprising a database of key words, phrases, and engaged to a software platform for electronically connecting the central hub to the control panel and the at least two communication devices; said first and second communication device having said software platform running on said device; wherein said second communication device generates a video communication request to said server, wherein said software identifies the second communication device and an electronic medical record connected to said software running on said second communication device; wherein said first communication device is notified of said video communication and said central hub generates a control panel, accessible on said first communication device, identifying the features of the second communication device; wherein the first and second communication device generate a video communication, and said control panel is utilized to optimize the sound and video quality by increasing or decreasing the sensitivity of microphone, increase or decrease the volume played by a speaker in each device, and increasing or decreasing the light contrast of the video camera to a pre-defined parameters.

20. The system of claim 19, wherein the control panel can manually modify one or more parameters of sound and video quality.

21. The system of claim 19 wherein the software is a mobile application.

22. The system of claim 19 further comprising a real time monitoring of the first communication device, said real time monitoring comprising optimizing the parameters of the first communication device, providing feedback to the provider to modify one or more actions, and optimizing parameters of the first communication device.

23. The system of claim 22, wherein the real time monitoring provides feedback on the control panel to automatically or manually modify one or more parameters on the first or second communication device.

24. The system of claim 19 further comprising a video overlay which is displayed on said control panel, said video overlay providing geolocation of the second communication device.

25. The system of claim 19 wherein said central hub is capable of recording voice or video, and said control panel capable for engaging recording status; and recording voice and video of instructions to a patient.

26. The system of claim 25, wherein the recording of voice and video of the instructions to a patient are transcribed from a recording to text.

27. The system of claim 19 wherein the control panel is engaged to control one or more features on the second communication device; wherein the control of one or more features engages with a gyroscope on said second communication device to orient said communication device into a desired position.

28. The system of claim 27, further engaging the accelerometer of the second communication device.

29. The system of claim 27 further engaging at least one camera on said second communication device.

30. The system of claim 27 wherein the second communication device generates sounds or visual cues for orienting the second communication device into said desired position.

31. The system of claim 19, wherein a communication is generated to a third communication device, based upon need or state licensing requirements to provide medical services.

32. The system of claim 19, wherein the central hub is capable of detecting speech and for identifying key words or phrases related to medical care; wherein identification of key words or phrases are combined with an electronic medical record (EMR) having codes corresponding to medical information is accessed by said server, and said key words or phrases and said EMR are utilized to generate a survey.

33. The system of claim 19, wherein the central hub is capable of detecting speech and for identifying key words or phrases related to medical care; wherein identification of key words or phrases are combined with an electronic medical record (EMR) having codes corresponding to medical information is accessed by said server, and wherein a coupon code is generated in response to the key words or phrases and said EMR.

34. The system of claim 33, further comprising a database stored on said central hub of coupon codes, wherein the key words or phrases and the EMR are compared to the database of said coupon codes for identifying coupon codes for deliver to the second communication device.

35. The system of claim 19; wherein the server generates an automatic message to the second communication device for scheduling a secondary call.

36. The system of claim 19; wherein a voice connection is first generated and wherein the control panel is engaged to modify the connection from a voice connection to a video connection; wherein the control panel and server generate a signal to the first communication device and the second communication device to activate a camera and engage video mode.

37. The system of claim 19, wherein the central hub is capable of detecting speech and for identifying key words or phrases related to medical care; wherein identification of key words or phrases are combined with an electronic medical record (EMR) having codes corresponding to medical information, accessed by said server, and said server further comprising a database comprising advertisements corresponding to one or more key words, phrases, or codes in an EMR, and wherein said key words or phrases, or EMR codes are compared to said database comprising advertisements, and wherein said advertisements are delivered to said second communication device.

38. A method of optimizing sound, and video parameters on a telehealth video call comprising:
a. Generating a video connection between a central hub, a control panel, and a first and second communication device;
b. Reviewing the parameters for sound, by comparing the received sounds from the first and second communication devices to a feedback level standard; comparing the video parameter to a predetermined video feed quality;
c. Modifying one or more of a microphone sensitivity, a video sensitivity, a camera zoom, a camera black and white balance, and speaker volume to modify the parameters;
d. Reviewing the modified parameters and compare to the feedback level standard and predetermined video feed quality; and
e. Proceeding with said video call, while repeating steps b and c automatically during said call to maintain said optimized settings.

39. The method of claim 38 wherein a parameter can be manually modified by the first or second communication device.

40. The method of claim 38 wherein the control panel can modify the second communication device manually.

41. A method of orienting a mobile device to a predetermined position for capturing an image comprising:
a. Connecting said mobile device to a central hub system; said central hub system comprising a central hub, a control panel, and a first communication device; providing access to said hardware and software controls on said mobile device, through a software application installed therein; wherein said software application engages said control panel to identify features of the mobile device capable of control;
b. Identifying a predetermined position on said control panel;
c. Generating a geolocation of said mobile device using one or more of the gyroscope, accelerometer, or camera;
d. Providing an oral or visual instruction to modify the present position of said mobile device to said predetermined position;
e. Providing an oral or visual instruction once said predetermined position is located;
f. Capturing an image from said camera at said location;
g. Uploading said image to said central hub.

42. The method of claim 41 wherein said image is provide for determining a medical diagnosis.

43. The method of claim 41 further engaging a flash or light on said mobile device when capturing said image from said camera.
